(19) United States
(12) Patent Application Publication
(10) Pub. No.: US 2024/0032833 A1
(43) Pub. Date: Feb. 1, 2024

(54) SYSTEMS AND METHODS FOR ASSESSMENT IN VIRTUAL REALITY THERAPY

(71) Applicant: Penumbra, Inc., Alameda, CA (US)

(72) Inventors: Joel Breton, Santa Rosa, CA (US); William Ka-Pui Yee, Alameda, CA (US)

(21) Appl. No.: 18/209,876

(22) Filed: Jun. 14, 2023

Related U.S. Application Data
(60) Provisional application No. 63/392,909, filed on Jul. 28, 2022.

(51) Int. Cl. A61B 5/16; A61B 5/0205; A61B 5/00; G16H 20/70

(52) U.S. Cl. CPC A61B 5/165; A61B 5/02055; A61B 5/748; A61B 5/7435; G16H 20/70; G16H 50/20

(57) ABSTRACT

Systems and methods may provide assessment tools such as assessment prompts, to solicit feedback from patients at regular intervals, track patient progress, and recommend VR activities. A VR therapy platform may use a VR assessment to request patient feedback related to conditions such as anxiety, depression, and/or pain. Based on patient-provided assessment responses, a VR therapy platform may categorize the patient responses and recommend one or more appropriate VR activities to help reduce the intensity of the patient's reported state(s). In some embodiments, VR activity recommendations may be based on patient-reported feedback and biometric measurements. In some embodiments, a VR therapy platform may compare a patient's assessment responses with biometric measurements taken at or around the same time, along with a patient's health history, to determine whether patient responses may be contradictory or biased, and thus potentially requiring adjustment prior to recommending VR activities.

### BACKGROUND OF THE DISCLOSURE

The present disclosure relates generally to virtual reality (VR) systems and more particularly to providing patient assessment in VR therapy or therapeutic activities or therapeutic exercises to engage a patient experiencing one or more health disorders.

### SUMMARY OF THE DISCLOSURE

Hospitals and therapists may request feedback from patients regarding their mental and physical status using a questionnaire form or other survey. Such feedback may shed light on how a patient feels mentally, physically, emotionally, and more. In some cases, doctors may diagnose potential mental or physical health disorders based, in part, on patient data collected from a questionnaire or assessment. Virtual reality (VR) systems may be used in various medical and mental-health related applications including various physical, neurological, cognitive, and/or sensory therapy. VR activities, exercises, videos, multimedia experiences, applications, and other content (referred to, together, as “activities”) may be used therapeutically, e.g., to help a patient improve his or her mental, physical, and/or emotional state. For instance, a patient who is feeling anxious may benefit from a VR meditation application or some patients with pain may improve their conditions over time from particular VR physical therapy exercises.

A VR platform may typically measure patient progress as merely completion of a list of activities, but there exists a need for more patient feedback data in VR to better determine current patient conditions and progress. Hospitals, doctors, and therapists need better, more frequent patient feedback regarding their mental, physical, and/or emotional conditions throughout a course of VR therapy—without dedicating staff to interview each patient at frequent intervals. With more feedback, appropriate VR activities may be recommended to appropriately address a patient's current conditions. Moreover, there exists a need for VR systems to collect and track patient-supplied feedback with measurements from patient biometrics, e.g., for comparison, corroboration, and analysis.

As discussed herein, a VR therapy platform may provide VR assessment tools such as survey questions and/or prompts in a VR interface, e.g., to solicit feedback from patients at regular intervals and track patient status and progress. A VR therapy platform may use VR assessment to request patient feedback related to conditions such as anxiety, depression, pain, and more. Based on patient-provided assessment responses, a VR therapy platform may categorize the patient responses and recommend one or more appropriate VR activities to help reduce the intensity of the patient's reported state(s). In some embodiments, VR activity recommendations may be based on patient-reported feedback and biometric measurements. A VR therapy platform may, for example, compare a patient's assessment responses with biometric measurements (assessed at or near the same time), along with a patient's health history, to determine whether patient assessment responses may be, e.g., corroborative or contradictory. If, for example, a bias is detected, assessment responses and/or values may require adjustment different weighting, e.g., prior to recommending appropriate VR activities.

### BRIEF DESCRIPTION OF THE DRAWINGS

FIG. 1 depicts an illustrative interface of a VR assessment platform, in accordance with embodiments of the present disclosure;

FIG. 2 depicts an illustrative interface of a VR assessment platform with directions, in accordance with embodiments of the present disclosure;

FIG. 3 depicts an illustrative interface of a VR assessment platform with an assessment prompt and data values, in accordance with embodiments of the present disclosure;

FIG. 4 depicts an illustrative interface of a VR assessment platform with an assessment prompt and data values, in accordance with embodiments of the present disclosure;

FIG. 5 depicts an illustrative interface of a VR assessment platform with an assessment prompt and data values, in accordance with embodiments of the present disclosure;

FIG. 6 depicts an illustrative interface of a VR assessment platform with an assessment prompt and data values, in accordance with embodiments of the present disclosure;

FIG. 7 depicts an illustrative interface of a VR assessment platform with an assessment prompt and data values, in accordance with embodiments of the present disclosure;

FIG. 8 depicts an illustrative interface of a VR assessment platform with an assessment prompt and data values, in accordance with embodiments of the present disclosure;

FIG. 9 depicts an illustrative interface of a VR assessment platform with an assessment prompt and data values, in accordance with embodiments of the present disclosure;

FIG. 10 depicts an illustrative scenario and interface of a VR voice control system, in accordance with embodiments of the present disclosure;

FIG. 11 depicts an illustrative interface of a VR assessment platform, in accordance with embodiments of the present disclosure;

FIG. 12 depicts an illustrative data structure for VR assessment scoring and categorizing, in accordance with embodiments of the present disclosure;

FIG. 13 depicts an illustrative data structure for VR assessment scoring and categorizing, in accordance with embodiments of the present disclosure;

FIG. 14 depicts an illustrative survey for compatibility of VR assessment scoring with hospital scoring, in accordance with embodiments of the present disclosure;

FIG. 15A depicts a flow chart of an exemplary process for VR assessment and VR activity recommendations, in accordance with embodiments of the present disclosure;

FIG. 15B depicts a flow chart of an exemplary process for VR assessment categorization, in accordance with embodiments of the present disclosure;

FIG. 16 illustrates exemplary components of a VR system, including biometric sensors, in accordance with some embodiments of the present disclosure;

FIG. 17 depicts a flow chart of an exemplary process for VR assessment with biometric data, in accordance with embodiments of the present disclosure;

FIG. 18 depicts a flow chart of an exemplary process for VR assessment with biometric data, in accordance with embodiments of the present disclosure;

FIG. 19 is an illustrative chart for collected biometric feedback during VR assessment, in accordance with some embodiments of the present disclosure;

FIG. 20 is an illustrative chart for collected biometric feedback during VR assessment, in accordance with some embodiments of the present disclosure;

FIG. 21A is a diagram of an illustrative system, in accordance with some embodiments of the disclosure;

FIG. 21B is a diagram of an illustrative system, in accordance with some embodiments of the disclosure;

FIG. 22 is a diagram of an illustrative system, in accordance with some embodiments of the disclosure;

FIG. 23 is a diagram of an illustrative system, in accordance with some embodiments of the disclosure; and

FIG. 24 is a diagram of an illustrative system, accordance with some embodiments of the disclosure.

### DETAILED DESCRIPTION OF THE DISCLOSURE

VR activities have shown promise as engaging therapies for patients suffering from a multitude of conditions, including various physical, neurological, cognitive, and/or sensory impairments, but more patient data may be needed. VR activities can be used to guide users in their movements while therapeutic VR can recreate practical exercises that may further rehabilitative goals such as physical development and neurorehabilitation. For instance, patients with physical and neurocognitive disorders may use therapy for treatment to improve, e.g., range of motion, balance, coordination, mobility, flexibility, posture, endurance, and strength. Physical therapy may also help with pain management. Some therapies, e.g., occupational therapies, may help patients with various impairments develop or recuperate physically and mentally to better perform activities of daily living and other everyday living functions. Additionally, cognitive therapy and meditative exercises, via a VR platform, may aid in improving emotional wellbeing and/or mindfulness. Through VR activities and exercises, VR therapy may engage patients better than traditional therapies, as well as encourage participation, consistency, and follow-through with a therapeutic regimen.

Generally, VR activities may use an avatar of the patient and animate the avatar in the virtual world. Using sensors in VR implementations of therapy allows for real-world data collection as the sensors can capture movements of body parts such as hands, arms, head, neck, back, and trunk, as well as legs and feet in some instances, for the system to convert and animate an avatar in a virtual environment. Such an approach may approximate the real-world movements of a patient to a high degree of accuracy in virtual-world movements and engage a patient. VR assessments, conducted in a VR interface, e.g., between VR activities, may too better engage a patient than pencil and paper or staff interviews.

Generally, hospitals and therapists may request feedback from patients regarding their mental and physical status using a questionnaire form or other survey, e.g., at the beginning or end of treatment. Such feedback may shed light on how a patient feels mentally, physically, emotionally, and more. In some cases, doctors may diagnose potential mental or physical health disorders using patient data collected from a questionnaire. Hospitals, doctors, and therapists need better, more frequent patient feedback regarding their mental, physical, and/or emotional conditions throughout a course of VR therapy. Likewise, current approaches in VR therapy platform typically only measure progress as completion of activities and scores. There exists a need for more patient feedback data to determine current patient conditions and progress.

The number of VR activities available to therapists and patients for exercise and therapy in a VR platform can be substantial. In some cases, VR activities are stored on the VR platform, e.g., in memory of a VR device such as the head-mounted display (“HMD”) and/or added over time. In some cases, VR activities may be downloaded from or accessed in the cloud on-demand and, e.g., there may be no apparent physical limits to how many VR activities that may be available to a therapist or patient. Finding the right VR activity is not always straightforward, even with titles, classifications, and/or descriptions available for searching and sorting. More and more VR activities are being developed to address specialized conditions with tailored VR exercises. With a variety of VR activities, comes a variety of exercises for therapy patients. However, not every exercise or activity is correct or properly suited for every patient. Moreover, general VR activity suggestions, e.g., based on patient profiles, will not account for a patient's current condition and what type of VR activities he or she respond to best at a given moment.

With more feedback, appropriate VR activities can be recommended to address a patient's current condition. Moreover, there exists a need for VR systems to collect and track patient-supplied feedback with measurements from patient biometrics, e.g., for comparison, corroboration, and analysis.

Some approaches to accessing VR activities may use an interface that allows users to efficiently navigate activity selections and easily identify activities that they may desire. An application which provides such guidance may be referred to as, e.g., an interface or a guidance application. For instance, via displays of an HMD, an interface may be presented as a graphical user interface, menus, buttons, boxes, lists, toggles, icons, slider bars, applications, tables, windows, and more. VR therapy platforms may provide user interfaces to facilitate identification and selection of a desired VR activity. Such an interface may also be utilized for other interactive portions, e.g., outside of activities. In some cases, voice commands and interaction may be used as part of a visual VR platform interface.

Interactive VR interfaces may utilize input from various sources for control, including remote controls, controllers, keyboards, a mouse, microphones, body sensors, video and motion capture, accelerometers, touchscreens, and others. In some approaches, head position, as measured by sensors in a HMD, may control a “gaze” cursor that can select buttons and interact with icons and menus in an interface of a VR platform. In some approaches, body sensors may track real world arm or hand movements to facilitate menu and interface navigation.

A VR system used in health care typically requires supervision, such as monitoring and/or guidance by a doctor or therapist. Generally, a health care provider may use a clinician tablet to monitor and control the patient's VR experience. A clinician tablet is typically in wired or wireless communication with an HMD and receives data in real time (or nearly real time). A VR system may be configured for in-person and/or remote observations.

To help a therapist, doctor, or supervisor of a VR system identify a patient's conditions and/or impairments, a VR therapy platform may incorporate additional data such as a patient's prior diagnoses and health information. Some VR systems may use, for example, a patient profile to store a patient's diagnosed conditions, therapy records, movement data, and activity performance data. Activities within VR applications may each have data stored to describe the goals and treatment in each activity or task. Prior to a therapist or supervisor initiating a therapy session, she should review patient impairments and impairments treated by the activity to ensure a good fit and avoid potentially injurious conflicts.

As discussed herein, a VR therapy platform may provide assessment tools such as survey prompts in a VR interface, e.g., to solicit feedback from patients at regular intervals and track patient status and progress. A VR therapy platform may use a VR survey to request patient feedback related to conditions such as anxiety, depression, pain, and more. Based on patient-provided survey responses, a VR therapy platform may categorize the patient responses and recommend one or more appropriate VR activities to help reduce the intensity of the patient's reported state(s).

In some embodiments, VR activity recommendations may be based on patient-reported feedback and biometric measurements. A VR therapy platform may, e.g., compare a patient's survey responses with biometric measurements, assessed at or near the same time, along with a patient's health history, to determine whether patient responses may be, e.g., corroborative or contradictory. If, for example, a bias is detected, survey responses and/or values may require adjustment and/or different weighting, e.g., prior to recommending VR activities.

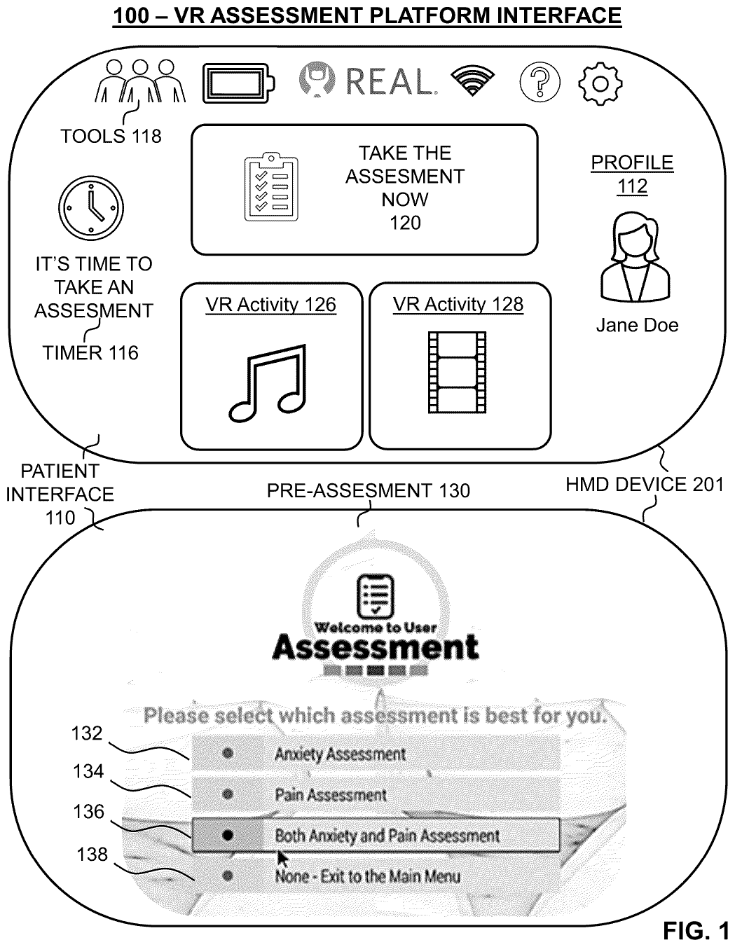

FIG. 1 depicts an illustrative interface of a VR assessment platform, in accordance with embodiments of the present disclosure. For instance, scenario 100 of FIG. 1 depicts a potential view of an interface, patient interface 110, in an HMD device 201, e.g., as seen by a patient. Scenario 100 features patient interface 110 in, e.g., two, sequential screens. In the first screen of patient interface 110, display comprises a toolbar at the top of the screen, tools 118, a profile option, profile 112, timer 116, VR activity 126, VR activity 128, and an option to take the assessment now 120. VR activities 126 and 128 maybe virtual reality activities, movies, music, and other content. In some embodiments, toolbar 118 may include settings for, e.g., battery strength, network connections, settings, help, and/or changing users. In some embodiments, patient interface 110 may include options and/or user interface elements to facilitate access to other VR activities.

In scenario 100, according to timer 116, it is time to take an assessment. In some embodiments, the option to take the assessment now 120 may only be available add certain times and/or intervals. In some embodiments, the assessment may be initiated automatically upon timer expiration. In some embodiments, and option to take the assessment now 120 may only appear when a timer expires. A timer may be set, for instance, atone or more regular intervals, such as every 5 minutes, 10 minutes, and/or 30 minutes. In some embodiments, a timer may be adjusted based on completion of one or more VR activities. For example, and assessment may be offered at a regular interval (e.g., every 10 minutes), as well as before and slash after each VR activity.

In patient interface 100, a patient may navigate using various inputs. For instance, a cursor, such as a dot, arrow, or crosshairs, may be used based on a patient's gaze and manipulated by head and neck movement to be directed at each icon and/or user interface element. Selecting, while using a gaze technique, may include holding the gaze at a fixed point while the cursor transforms into a meter or counter for, e.g., 3 seconds, and sufficient time passes to ensure the patient meant to hold the gaze cursor on the UI element for selection. For example, a patient may gays at the user interface element (e.g., icon and/or box) to select the option to take the assessment now 120. In some embodiments, done VR platform may use one or more control methods, such as remote controls, controllers, keyboards, a mouse, microphones, body sensors, video and motion capture, accelerometers, touchscreens, and others.

In the second screen of scenario 100, the VR assessment begins with, e.g., pre-assessment 130. In pre-assessment 130, the patient may be welcomed, and options may be presented for one or more assessments, such as anxiety assessment 132, pain assessment 134, both anxiety and pain assessment 136, and none—exit to the main menu 138. In some embodiments, the proper assessment for a particular patient maybe preselected, e.g., by the doctor or therapist. In some embodiments, the proper assessment for a particular patient may be preselected automatically based on the patient profile 112. In some embodiments, other assessments may be available. In some embodiments, different combinations of, e.g., anxiety, pain, and/or depression assessments may be offered. In scenario 100, one of the options may be selected by moving a cursor, e.g., using gaze and/or another control mechanism.

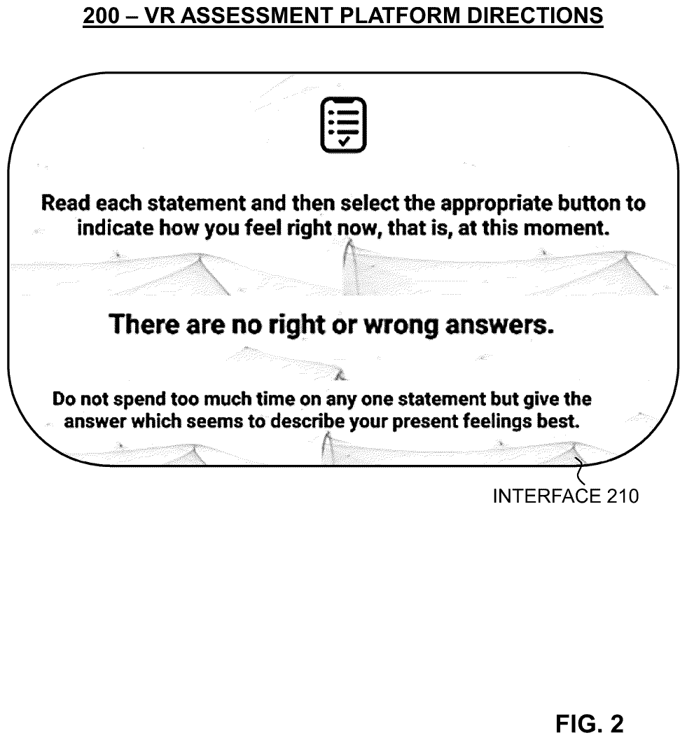

FIG. 2 depicts an illustrative interface of a VR assessment platform with directions, in accordance with embodiments of the present disclosure. Scenario 200 depicts interface 210, which presents the VR assessment platform directions. Interface 210, for example, may be presented in a patient's HMD 201. VR assessment platform directions make comprise one or more instructions and or warnings. For instance, interface 210 may present instructions such as one or more of: “Read each statement and then select the appropriate button to indicate how you feel right now, that is, at this moment,” “There are no right or wrong answers,” and “Do not spend too much time on anyone statement but give the answer which seems to describe your present feelings best.” Generally, a VR assessment may comprise several questions and/or prompts regarding how the patient feels at this particular time.

The VR assessment platform may, for instance, attribute values to each selected response in the assessment and, based on the total value of the responses, may provide some feedback to the patient (and medical team) and/or recommendations for VR activities for the patient to, e.g., help improve how the patient is currently feeling. The VR assessments can be an important tool in measuring whether VR therapy is helping the patient improve how he or she is feeling. In some embodiments, an assessment may be given at regular intervals to evaluate progress on how the patient is feeling before, during, and/or after VR therapy. In some embodiments, an assessment may be given at before, during, and/or after each VR activity to, e.g., evaluate progress on how the patient is feeling throughout a VR therapy session.

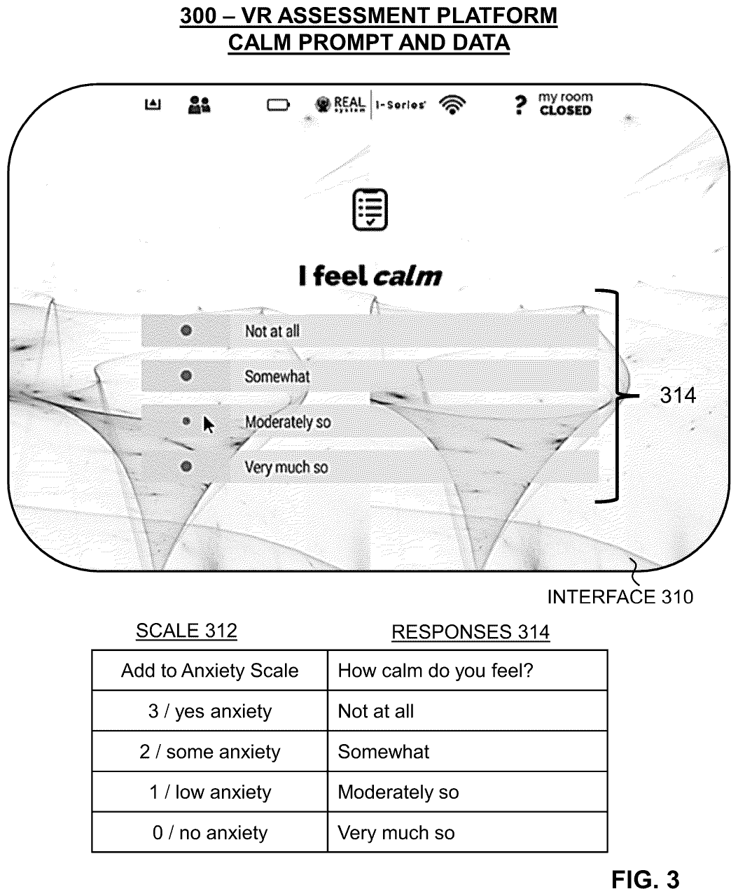

FIG. 3 depicts an illustrative interface of a VR assessment platform with an assessment prompt and data values, in accordance with embodiments of the present disclosure. Generally, each VR assessment prompt is designed to collect and score how a patient is feeling, e.g., in order to statistically analyze the scores together and/or individually. A VR assessment may prompt a patient with a question or statement, ask the patient to select a response, and assign a value to the response. Scenario 300 presents a first prompt in a VR assessment platform. Interface 310, for example, may be presented in a patient's HMD 201. Interface 310 features an assessment prompt relating to how calm a patient may feel at this particular time. For instance, interface 310 may prompt, “I feel calm” and present response options 314 of, e.g., “Not at all,” “Somewhat,” “Moderately so,” or “Very much so.” In scenario 300, each of the responses 314 has a corresponding score on and anxiety scale 312. As a patient progresses through the VR assessment and answers each assessment prompt, a score may be attributed to each response so that sum of the response scores (e.g., in each category) may be used for evaluation. For instance, with the cursor selecting “Moderately so,” a score of “1,” e.g., “low anxiety,” would be added to the patient's anxiety score. In scenario 300, a response of “Very much calm” would yield a score of 0 to be added to the anxiety scale for the patient. In scenario 300, a response of “Somewhat calm” would yield and anxiety scale addition of 2, showing some anxiety by this response. If a patient were feeling “Not calm at all,” then an addition of 3 may be added to the patient's anxiety scale as he or she progresses through the assessment prompts. Scenario 300, an anxiety prompt and selection interface, may appear, for example, as part of an anxiety assessment or as part of one or more other assessments like anxiety, depression, pain, and/or any combination thereof.

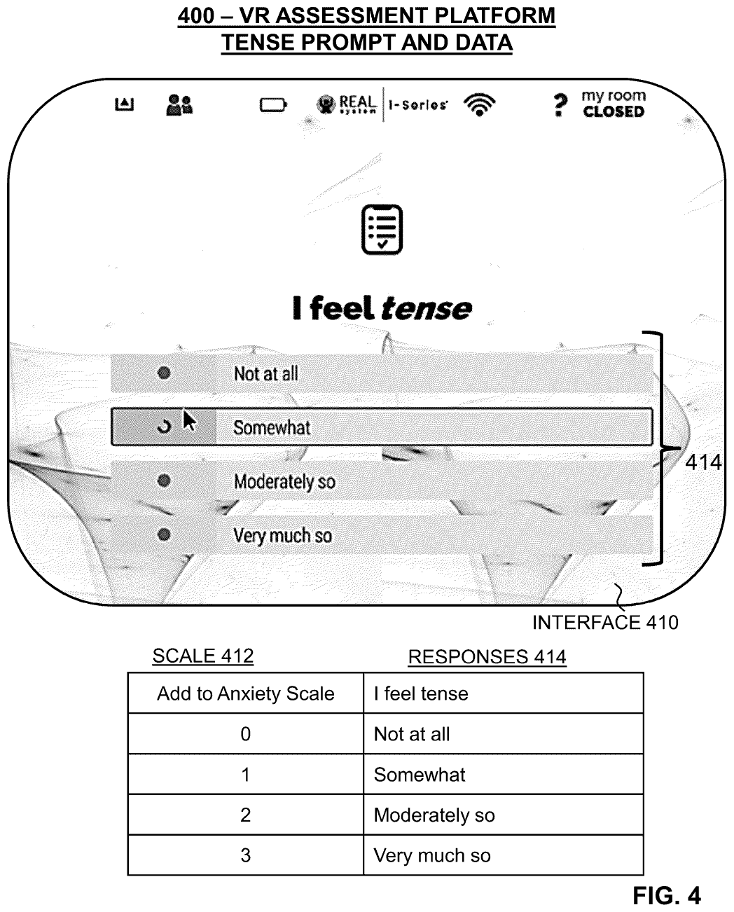

FIG. 4 depicts an illustrative interface of a VR assessment platform with an assessment prompt and data values, in accordance with embodiments of the present disclosure. Scenario 400 presents another prompt in an interface of a VR assessment platform. Interface 410, for example, may be presented in a patient's HMD 201. Interface 410 includes responses 414 and a corresponding scale 412. For instance, with the prompt “I feel tense,” responses 414 include “Not at all,” “Somewhat,” “Moderately so,” and “Very much so,” with anxiety scale scores 412 of 0, 1, 2, and 3, respectively. The anxiety scale values corresponding to responses for a prompt about feeling tense may be different (e.g., inverted) from when compared to anxiety scale values for responses to a prompt regarding feeling calmness. In scenario 400, the cursor may select “Somewhat” to express, e.g., that the patient is feeling somewhat calm, so a value of 1 would be added to the patient's anxiety scale score.

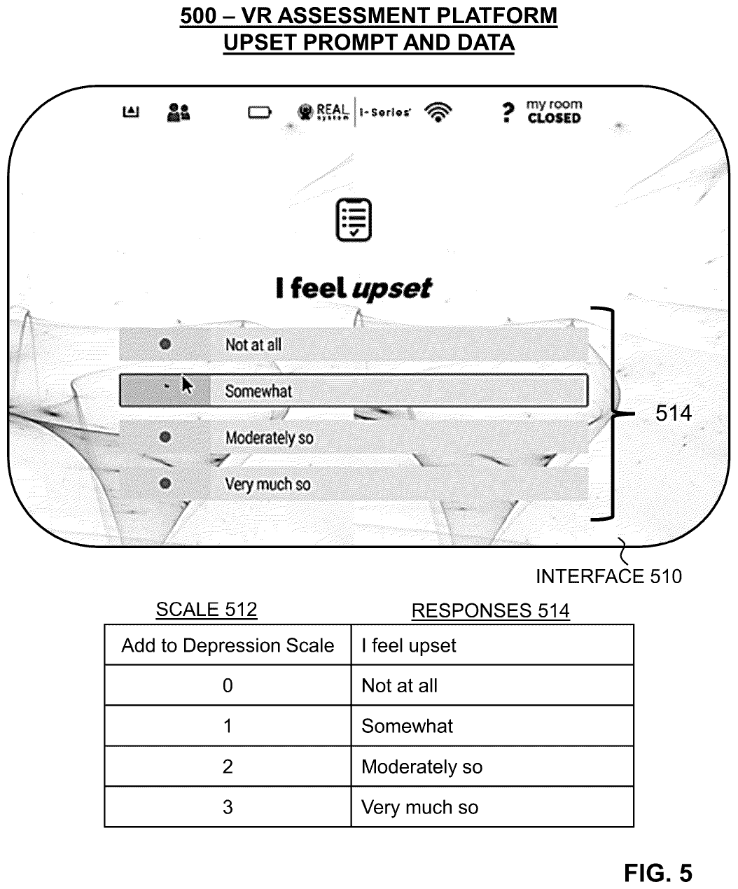

FIG. 5 depicts an illustrative interface of a VR assessment platform with an assessment prompt and data values, in accordance with embodiments of the present disclosure. Scenario 500 presents another prompt in an interface of a VR assessment platform. Scenario 500 may be a prompt in a depression assessment. Interface 510, for example, may be presented in a patient's HMD 201. Interface 510 includes responses 514 and a corresponding scale 512. In scenario 500, the scale is a depression scale for each response, and the score would be added to a depression scale score for evaluation of depression. For instance, with the prompt “I feel upset,” responses 514 include “Not at all,” “Somewhat,” “Moderately so,” and “Very much so,” with depression scale scores 512 of 0, 1, 2, and 3, respectively. Scenario 500, a depression prompt and selection interface, may appear, for example, as part of a depression assessment or as part of one or more other assessments like anxiety, depression, pain, and/or any combination thereof.

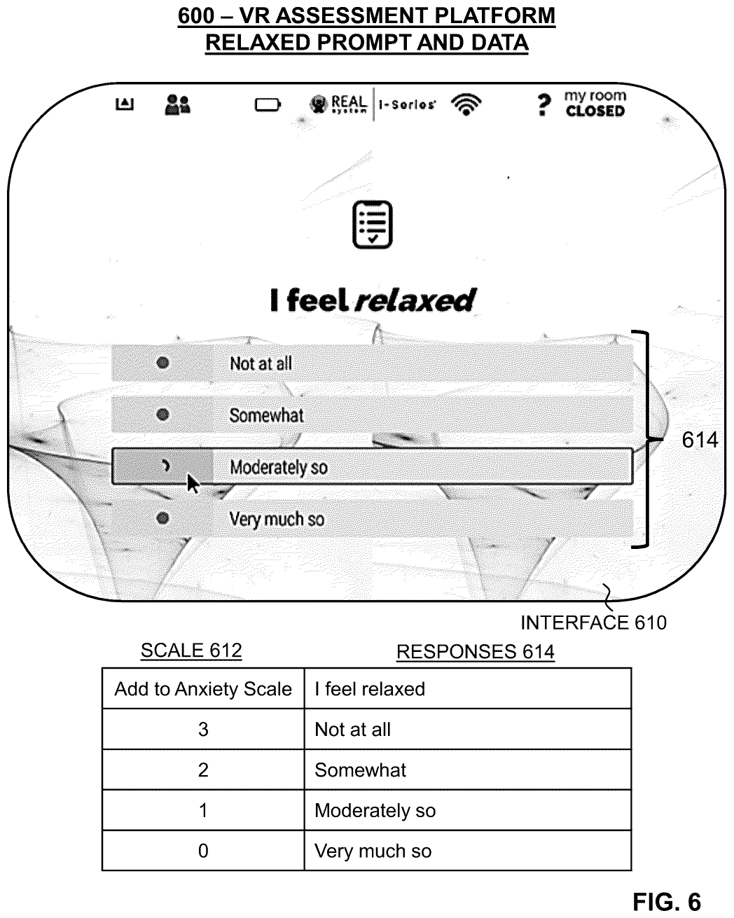

FIG. 6 depicts an illustrative interface of a VR assessment platform with an assessment prompt and data values, in accordance with embodiments of the present disclosure. Scenario 600 presents another prompt in an interface of a VR assessment platform. Interface 610, for example, may be presented in a patient's HMD 201. Interface 610 includes responses 614 and a corresponding scale 612. For instance, with the prompt “I feel relaxed,” responses include “Not at all,” “Somewhat,” “Moderately so,” and “Very much so,” with anxiety scale scores 612 of 3, 2, 1, and 0, respectively. Not feeling “relaxed” may indicate several potential issues, e.g., that may be better identified with other assessment questions. In some embodiments, a prompt like “I feel relaxed” might be used in multiple scales, such as an anxiety scale, a depression scale, and/or a pain scale.

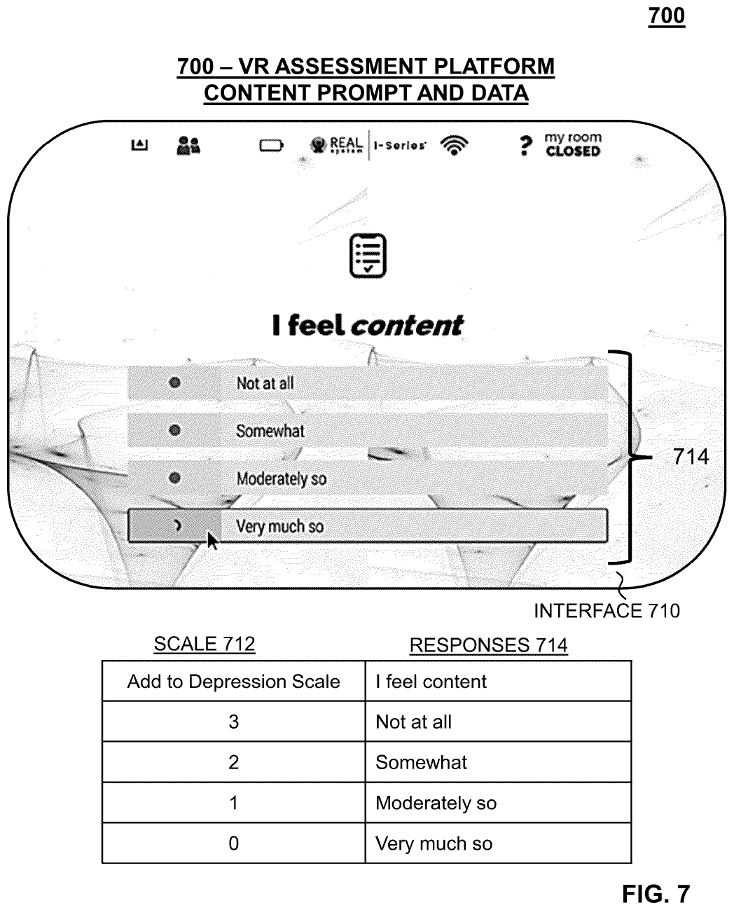

FIG. 7 depicts an illustrative interface of a VR assessment platform with an assessment prompt and data values, in accordance with embodiments of the present disclosure. Scenario 700 presents another prompt in an interface of a VR assessment platform. Interface 710, for example, may be presented in a patient's HMD 201. Interface 710 includes responses 714 and a corresponding scale 712. For instance, with the prompt “I feel content,” responses include “Not at all,” “Somewhat,” “Moderately so,” and “Very much so,” with depression scale scores 712 of 3, 2, 1, and 0, respectively.

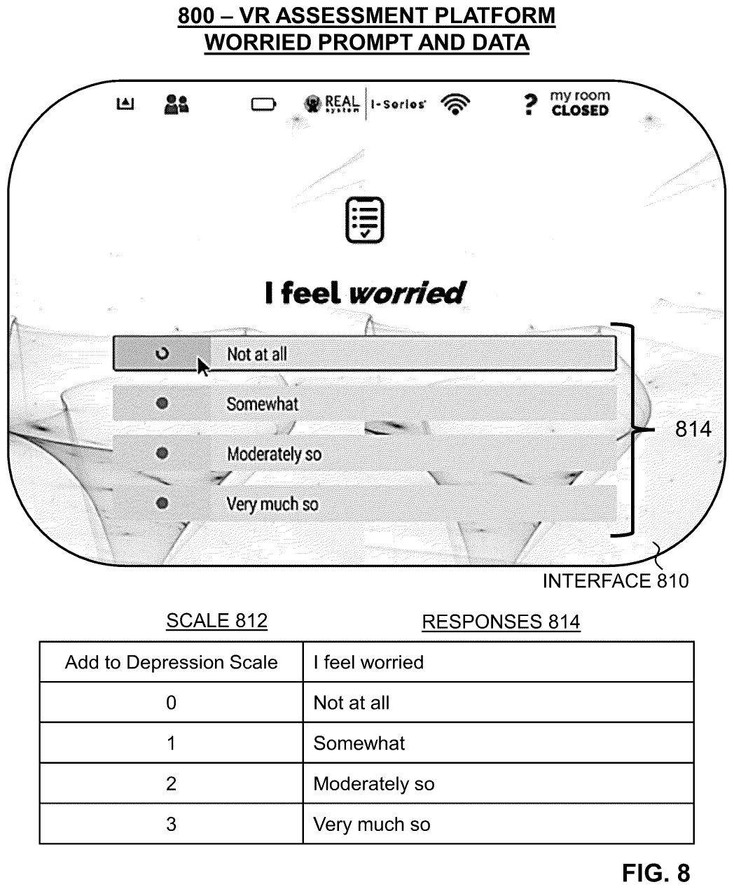

FIG. 8 depicts an illustrative interface of a VR assessment platform with an assessment prompt and data values, in accordance with embodiments of the present disclosure. Scenario 800 presents another prompt in an interface of a VR assessment platform. Interface 810, for example, may be presented in a patient's HMD 201. Interface 810 includes responses 814 and a corresponding scale 812. For instance, with the prompt “I feel upset,” responses 814 include “Not at all,” “Somewhat,” “Moderately so,” and “Very much so,” with depression scale scores 812 of 0, 1, 2, and 3, respectively. In some embodiments, a question about “worrying” may prompt, e.g., “Worrying thoughts go through my mind:” and offer responses such as “Only occasionally,” “From time to time, but not too often,” “A lot of the time,” and “A great deal of time,” with depression scale scores of 0, 1, 2, and 3, respectively.

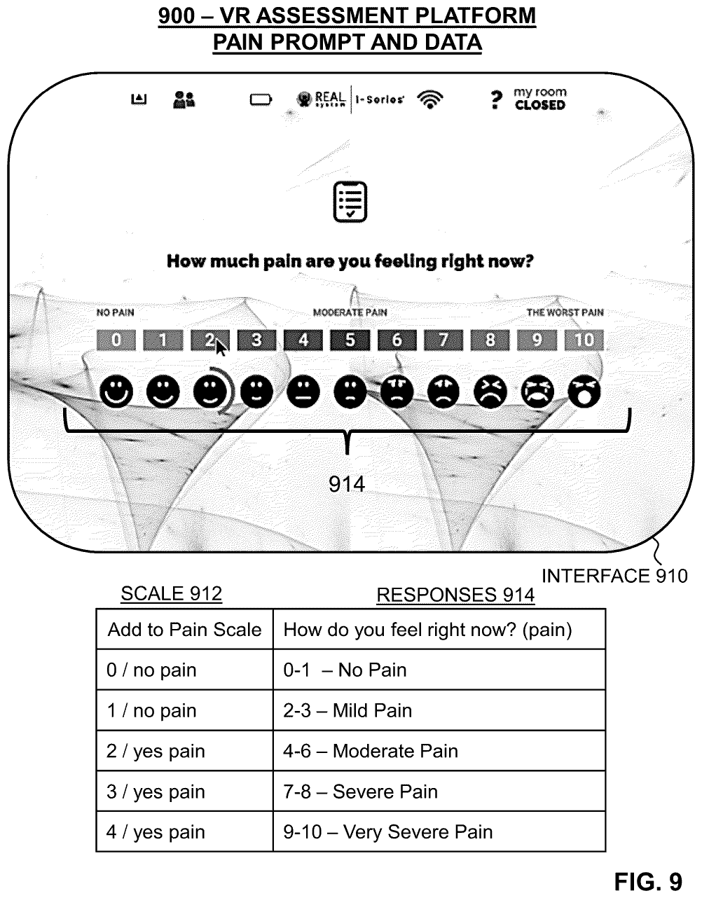

FIG. 9 depicts an illustrative interface of a VR assessment platform with an assessment prompt and data values, in accordance with embodiments of the present disclosure. Scenario 900 presents another prompt in an interface of a VR assessment platform. Interface 910, for example, may be presented in a patient's HMD 201. Interface 910 includes responses 914 and a corresponding scale 912. In scenario 900, a patient is asked “How much pain are you feeling right now?” and given a scale from 0 to 10. In this case, zero means no pain and 10 means the worst pain, while five would indicate moderate pain. In scenario 900, a selected response score is converted to a pain scale score of about half (e.g., rounded down) of the response score. Here, the pain scale of 0 to 4 stays consistent with the other scales such as anxiety or depression. In some embodiments, scale 912 may directly correlate with the on-screen pain scale to be selected by the patient, e.g., 0 to 10, and normalized and/or weighted during further analysis. In some embodiments, a pain scale may be, e.g., from 0 to 9, 1 to 10, or 0-100. Scenario 900, a pain score prompt and selection interface, may appear, for example as part of a pain assessment or as part of one or more other assessments like anxiety, depression, pain, and/or any combination thereof.

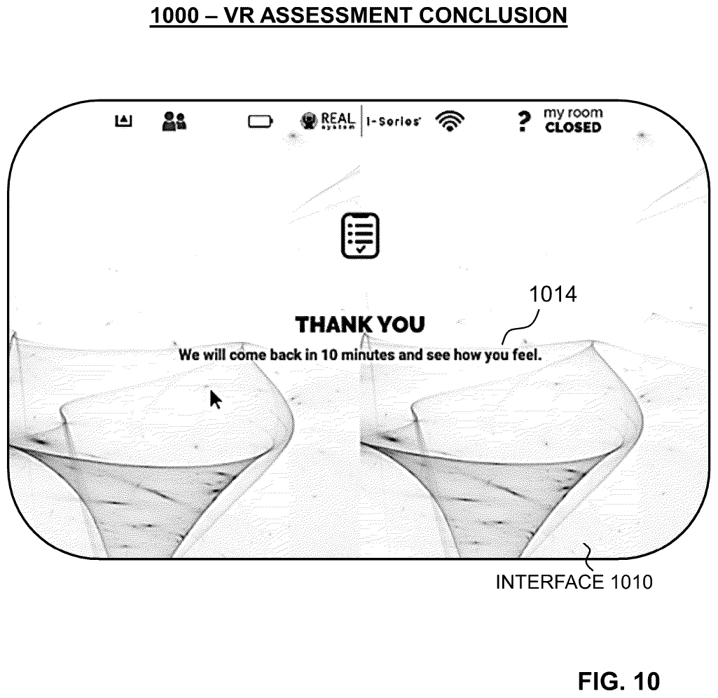

FIG. 10 depicts an illustrative scenario and interface of a VR voice control system, in accordance with embodiments of the present disclosure. Scenario 1000 presents another interface screen. Interface 1010, for example, may be presented in a patient's HMD 201. For instance, scenario 1000 may present interface 1010 after a session of VR assessment has concluded. Interface 1010 presents a thank you message and instructs the patient, in message 1014, that “We will come back in 10 minutes and see how you feel.” In some embodiments, message 1014 may indicate a different schedule for the next VR assessment. For instance, message 1014 may indicate a next assessment may be in 20 minutes or after the patience next VR activity (or multiple activities). In some embodiments, at this point, a VR assessment platform may analyze the patient's responses and perform one or more forms of statistical analysis on the collected data.

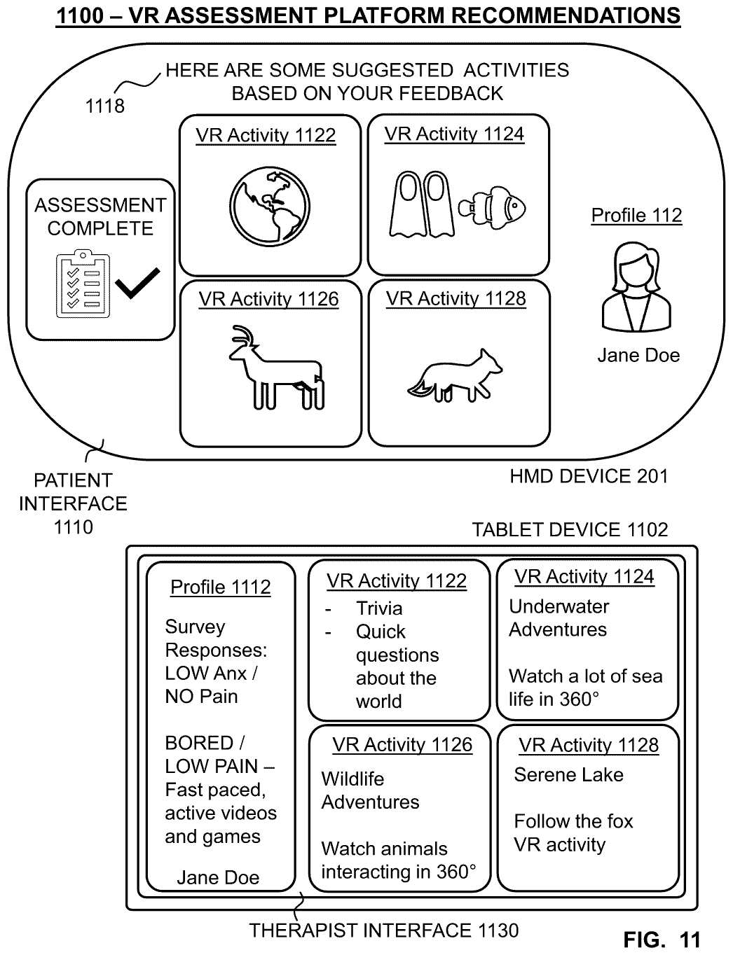

FIG. 11 depicts an illustrative interface of a VR assessment platform, in accordance with embodiments of the present disclosure. Scenario 1100 presents another interface screen, e.g., similar to scenario 100 or FIG. 1. Scenario 1100 depicts patient interface 1110 and therapist interface 1130, with patient interface 1110 appearing on HMD device 201 and therapist interface 1130 featured on (supervisor) tablet device 1102. In some cases, a therapist device 1102 (and/or a supervisor) may not be needed and therapist device 1120 may only be depicted to indicate additional data not necessarily revealed to a patient. Patient interface 1110 may offer some suggested activities 1118 based on the patient's assessment responses. For instance, patient interface 1110 indicates that the assessment is complete and suggests (e.g., based on the response values) some appropriate VR activities such as trivia 1122, underwater adventures 1124, wildlife adventures 1126, and serene lake 1128. Therapist interface 1130 offers descriptions on the suggested VR activities 1118 individually as well as data on the patient profile 1112 including survey responses and categorization of the survey response data. On therapist interface 1130, profile 1112 reflects that the survey responses indicate low anxiety and no pain, which may be categorized as “Bored” with “Low Pain” (or no pain). Therapist interface 1130 also indicates that patients categorized with “Bored” and “Low Pain” (e.g., or no pain) may need VR activities such as fast paced and active videos and games, e.g., based on the patient's survey responses. In some embodiments, VR activities 1122, 1124, 1126, and 1128 may be recommended just based on a categorization of survey responses. In some embodiments, VR activities maybe suggested or recommended based on statistical analysis of survey responses. In some embodiments, VR activities may be suggested or recommended based on some combination of survey responses, data analysis, and patient profile(s).

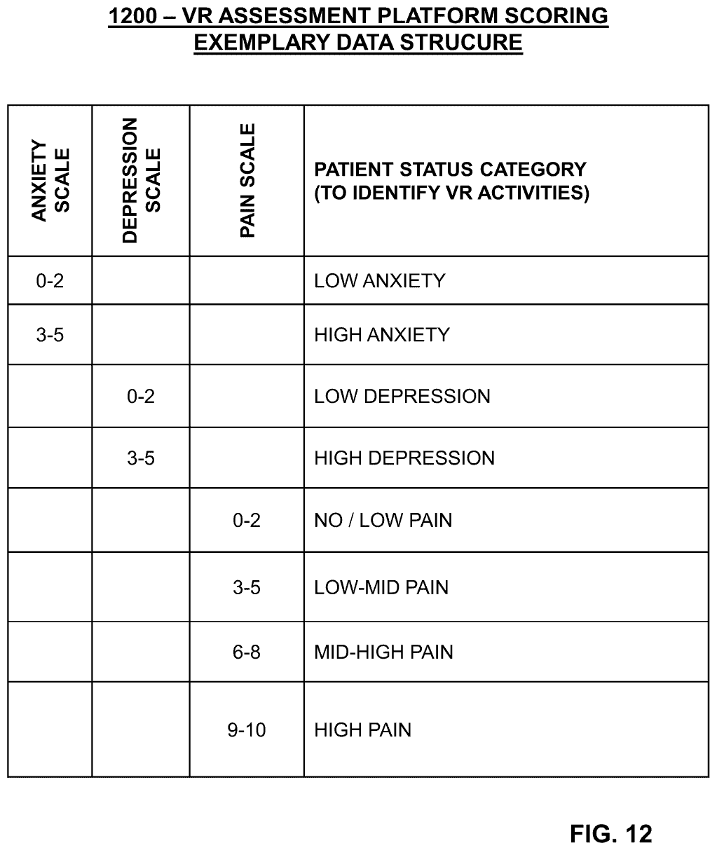

FIG. 12 depicts an illustrative data structure for VR assessment scoring and categorizing, in accordance with embodiments of the present disclosure. In some embodiments, VR assessment platform scoring may be in one or more patient status categories based on assessments and/or more scales, e.g., anxiety, depression, and/or pain. In scenario 1200, an average in an assessment category may be used to categorize a patient status. For instance, based on a patient's responses in an anxiety assessment the sum of the anxiety scores may be divided by the number of responses provided (or prompts given) to produce a mean and/or average for the anxiety scale. In scenario 1200, if an anxiety scale score is between 0 and 2 the patient is considered to have “low anxiety” and, e.g., VR activities associated with low anxiety should be recommended. If anxiety assessment responses yield an anxiety scale score between 3 and 5, VR activities associated with patients with high anxiety should be suggested. Further, in scenario 1200, a depression scale score between zero and two indicates low depression and a depression scale score between 3 and 5 indicates high depression. Regarding pain in scenario 1200, the scale is left as 0-10, so, for instance, a pain scale score between 0 and 2 may indicate no or low pain, a pain scale score between 3 and 5 may indicate low- to mid-level pain, a pain scale score between 6 and 8 may indicate mid- to high-level pain, and a pain scale score of 9 and 10 may indicate high pain. In some embodiments, each scale may be normalized and/or adjusted, e.g., based on the number of potential categories able to be assigned to a patient. In some embodiments, other mathematical and statistical measurements may be used to determine a scale score from the response data set, e.g., sum, mean, mode, median, maximum, minimum, standard deviation, etc.

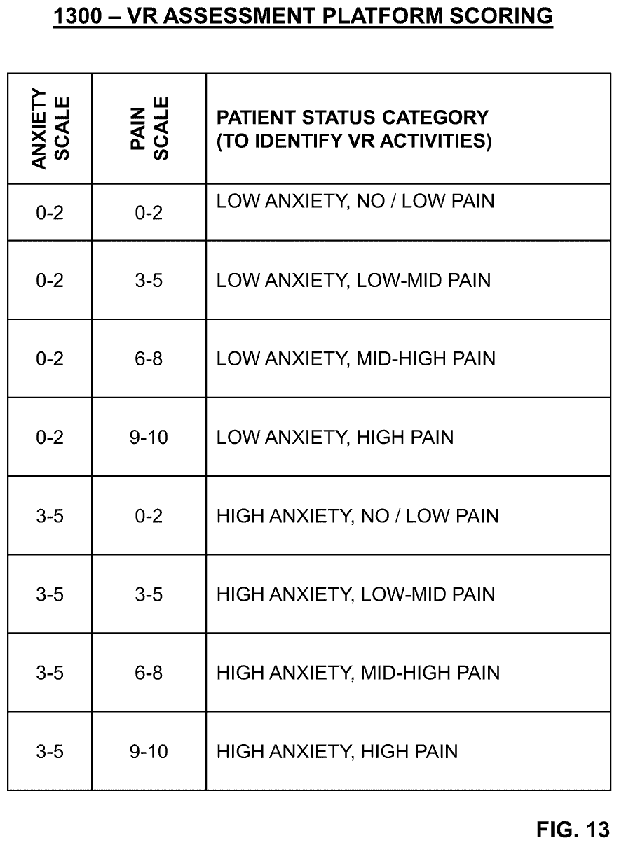

FIG. 13 depicts an illustrative data structure for VR assessment scoring and categorizing, in accordance with embodiments of the present disclosure. In some embodiments, VR assessment platform scoring may be in combinations of categories, e.g., based on assessments on scales for, e.g., anxiety, depression, and/or pain. In scenario 1300, an average in each assessment category, e.g., anxiety and pain, may be used to categorize a patient for recommendation of VR activities. Scenario 1300 uses similar scales as scenario 1200 for anxiety and pain, however, they are no longer separated. Scenario 1300 features an anxiety scale between 0 and 2 and a pain scale between 0 and 10. Each combination of category for anxiety and pain may be included. For instance, a patient whose responses in an anxiety assessment yield an anxiety scale score between 0 and 2 and responses in a pain assessment yield a pain scale score between 6 and 8 may be categorized as low anxiety and mid- to high-level pain. A patient whose responses in an anxiety assessment yield an anxiety scale score between 3 and 5 and responses in a pain assessment yield a pain scale score between 3 and 5 may be categorized as high anxiety and low- to mid-level pain. In some embodiments, each scale may be adjusted based on, e.g., the desired granularity of the categories, as well as categories available for classification and VR activity recommendations. In some embodiments, each scale may be normalized and/or adjusted, e.g., based on the number of potential categories able to be assigned to a patient. In some embodiments, other mathematical and statistical measurements may be used to determine a scale score from the response data set, e.g., sum, mean, mode, median, maximum, minimum, standard deviation, etc.

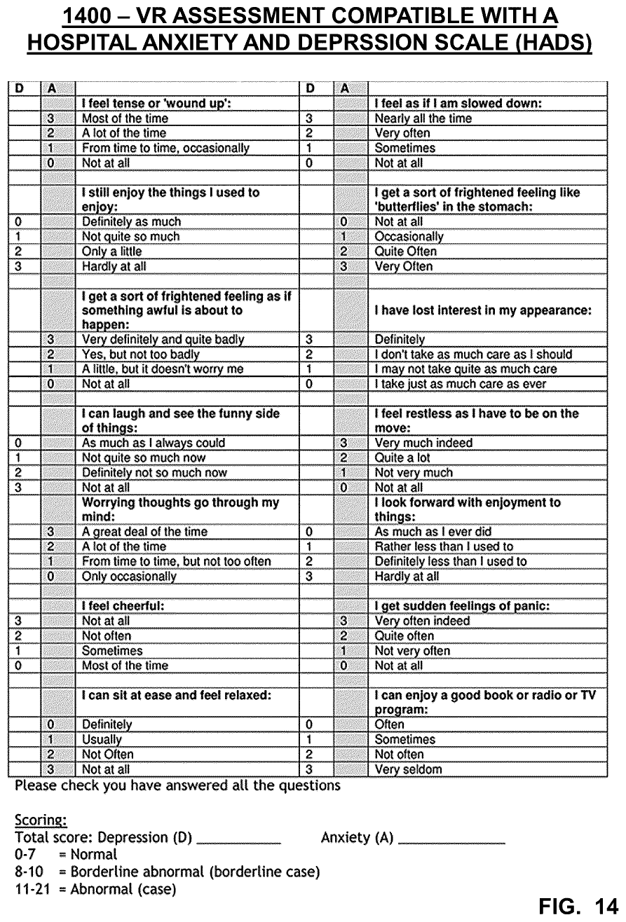

FIG. 14 depicts an illustrative survey for compatibility of VR assessment scoring with hospital scoring, in accordance with embodiments of the present disclosure. Scenario 1400 features depression and anxiety scales that are compatible with the Hospital Anxiety and Depression Scale (HADS). Some embodiments may use a scale compatible with adds in order to more easily interface with current hospital and therapeutic assessments. For instance, some questions from the HADS survey maybe incorporated into a VR assessment platform survey or questionnaire. The HADS survey of Scenario 1400 uses a scoring system utilizing a total score for each of depression scales and anxiety scales, e.g., with a result of “Normal” indicated by a score of 0 to 7, a score of 8 to 10 indicating “Borderline abnormal,” and scores of 11 to 21 indicating “Abnormal,” in each of depression and anxiety, respectively. While a HADS questionnaire may typically be administered by a nurse, doctor, or other hospital staff and then calculated by hand, a VR assessment platform may automatically provide similar survey questions, e.g., at regular intervals (and/or based on occurrence of certain events), collect and record data, categorize patients based on scores, and offer recommendations—e.g., without staff involvement. A staff member administering a HADS test every 10 minutes may be tedious, cause patient frustration, and generally inefficient, however, a VR assessment may be quicker and more fruitful.

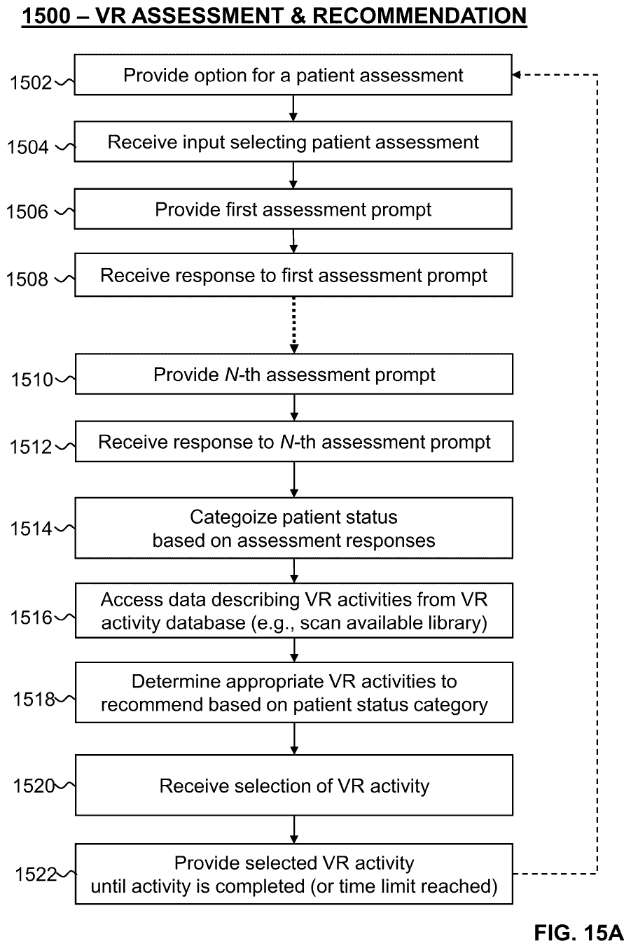

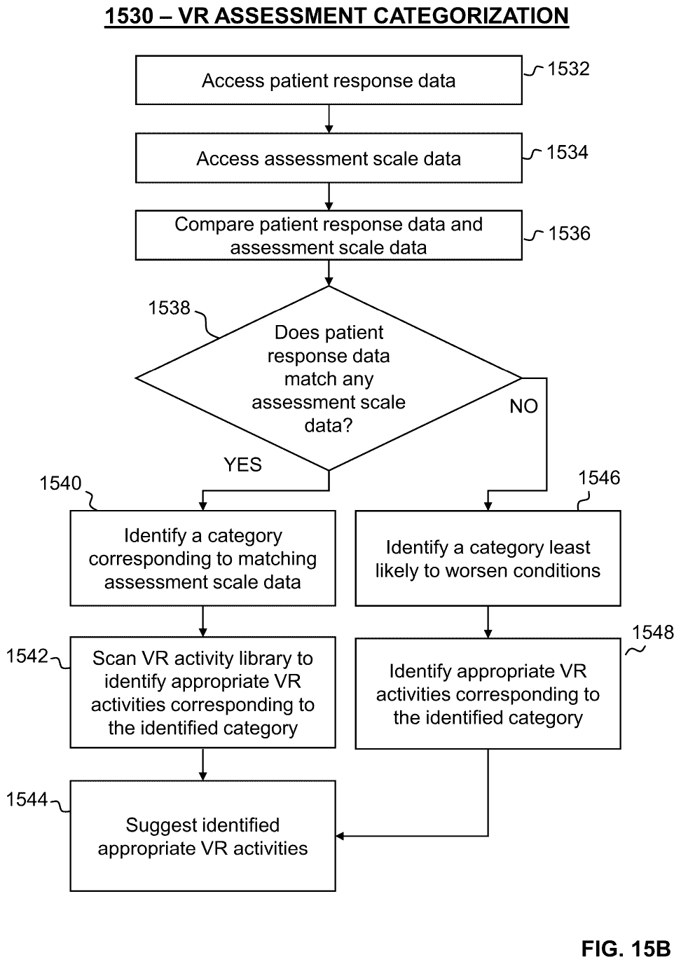

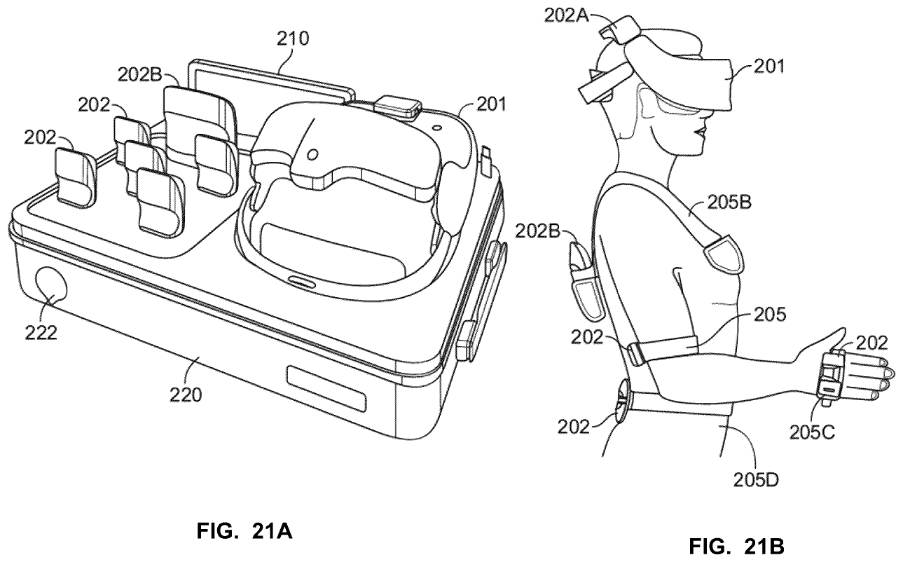

FIG. 15A depicts a flow chart of an exemplary process for VR assessment and VR activity recommendations, in accordance with embodiments of the present disclosure. VR assessment and recommendation may be carried out by one or more VR engines, e.g., as part of a VR platform running on VR hardware and/or network servers as depicted in FIGS. 21-24. At step 1502, a VR engine may provide an option for a patient assessment. For instance, in FIG. 1, “take the assessment now” button 120 may be an option for a patient assessment. At step 1504, the VR engine may receive input selecting to begin the patient assessment. Such input may be from, e.g., a cursor, a controller, a gaze input, accelerometer and/or motion sensors, a remote control, voice command, etc. In some embodiments, the VR engine may prompt a patient to select a particular assessment, e.g., an anxiety assessment, a pain assessment, and/or a depression assessment. Some embodiments may offer multiple assessments together. For instance, pre-assessment screen 130 of FIG. 1 features options for one or more assessments, such as anxiety assessment 132, pain assessment 134, both anxiety and pain assessment 136, and none—exit to the main menu 138. Some embodiments may have assessments preselected by a therapist.

At step 1506, as part of the survey and/or assessment, the VR engine may provide a first assessment prompt. A first prompt may be, for example, “I feel calm” as depicted in FIG. 3, with response options 314 such as “Not at all,” “Somewhat,” “Moderately so,” and “Very much so.” At step 1508, the VR engine may receive a patient response to the first assessment prompt. For example, a patient may respond to “I feel calm” with “Moderately so” as depicted in FIG. 3, which has a corresponding anxiety scale value of 1. Several prompts and/or questions may be provided during the assessment session, and many responses received and recorded. For instance, FIGS. 3-9 and 14 depict sample prompts and responses, for one or more assessments. At step 1510, the VR engine may provide an Nth assessment prompt. At step 1512, the VR engine may receive a patient response for the Nth assessment prompt. After all the assessment prompts have been provided, and the responses have been collected, at step 1514, the VR engine may categorize the patient's status based on the assessment responses. For instance, data structure 1200 in FIG. 12 uses the average value of the survey responses to categorize the patient status. Data structure 1300 in FIG. 13 uses the average value of the anxiety assessment responses combined with at least one pain assessment response to identify categories of patients with low or high anxiety combined with one of no, low, medium, or high pain levels.

At step 1516, the VR engine may access data describing VR activities from a VR activity database. For example, this may include scanning the available VR activities in a library. At step 1518, the VR engine may determine one or more appropriate VR activities to recommend based on the patient status category. For instance, based on search VR activity data and the categorized patient status, a match with one or more VR activities that are appropriate with the patient status category may be determined. Scenario 1100 of FIG. 11 presents suggested activities 1118 related to “BORED/LOW PAIN”, e.g., based on a patient categorization of low anxiety and no pain.

At step 1520, the VR engine may receive a patient selection of a VR activity, e.g., one of the recommended VR activities. Generally, upon selection of a VR activity, the VR activity will be provided to the patient for consumption and/or performance. For instance, a trivia game may be provided, or a 360-degree underwater video may be provided, and/or an interactive VR activity with a digital fox playing games near a lakeside forest may be provided, e.g., upon selection. After the activity is over, or a predetermined time limit is reached, at step 1522, the VR engine ceases to provide the VR activity (or pauses it) and the next assessment is provided at step 1502. By repeating the assessments at regular intervals or after each activity, the VR platform may monitor for improvements in patient responses and/or patient status. Again, a VR assessment can efficiently solicit patient responses and assess change in patient status. If improvement is not detected during or after an activity, the activity may be changed, and data may be recorded so that the activity may not be recommend as highly (or at all) for this patient (or similar patients).

FIG. 15B depicts a flow chart of an exemplary process for VR assessment categorization, in accordance with embodiments of the present disclosure. VR assessment and categorization may be carried out by one or more VR engines, e.g., as part of a VR platform running on VR hardware and/or network servers as depicted in FIGS. 21-24. At step 1532, a VR engine accesses patient response data. At step 1534, the VR engine accesses assessment scale data. At step 1516, the VR engine compares patient response data to the assessment scale data. At step 1538, the VR engine determines whether the patient response data matches any of the assessment scale data.

If the VR engine determines that the patient response data matches a portion of the assessment scale data then, at step 1540, the VR engine identifies a category corresponding to the matching assessment scale data. At step 1542, the VR engine scans the VR activity library to identify appropriate VR activities corresponding to the identified category. In some embodiments, the VR engine may determine if other VR activity metadata, e.g., titles, descriptions, categories, keywords, genres, styles, types, etc. match characteristics associated with a patient status category. At step 1544 the VR engine suggests identified appropriate VR activities.

If the VR engine determines that the patient response data does not match any of the assessment scale data then, at step 1546, the VR engine identifies a category least likely to worsen conditions. In some embodiments, this may be a default category leading to the least strenuous and most uplifting VR activities. In some embodiments, this may be the closest category, e.g., based on the response data and the assessment scale data. At step 1548, the VR engine identifies appropriate VR activities corresponding to the identified category. In some embodiments, the VR engine may have to scan the VR activity library but generally there may be default easy VR activities available. At step 1544 the VR engine suggests identified appropriate VR activities.

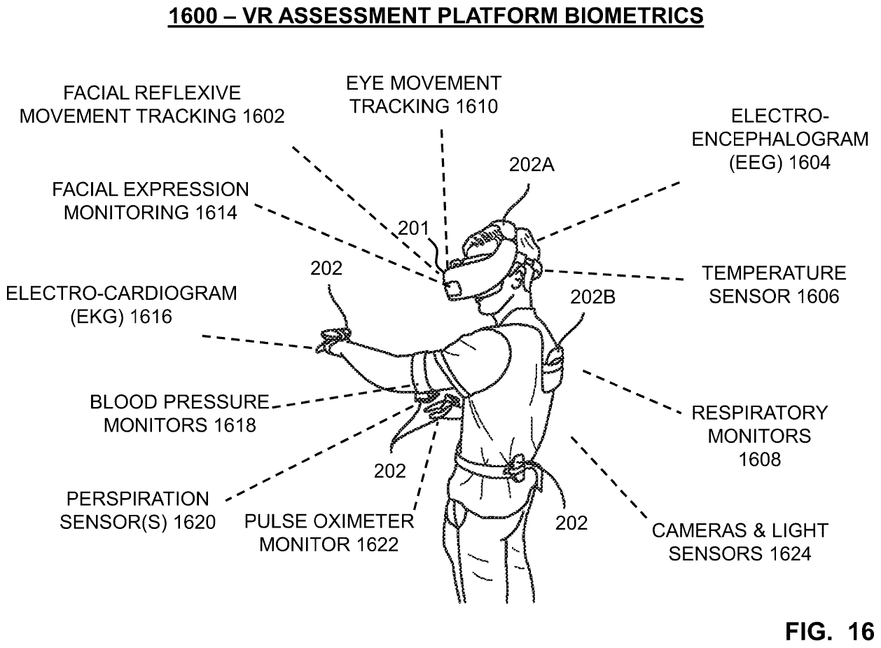

FIG. 16 illustrates exemplary components of a VR system, including biometric sensors, in accordance with some embodiments of the present disclosure. In some embodiments, patient-reported responses may not be the only input. Biometric data, such as data measured by biometric sensors like the devices depicted in FIG. 16, may be taken at various points during VR activities and VR therapy, generally. For instance, a patient's heart rate and/or blood pressure may be measured at a predetermined interval and/or at certain points of therapy to track whether a patient's emotional state is improving. In some embodiments, a biometric value may be recorded at the beginning of and/or end of, e.g., each VR activity. For example, a heart rate baseline may be set at the beginning of therapy and monitored at intervals for comparison to determine whether each VR exercise is helping (or exacerbating) the patient's heart rate. Similarly, perspiration sensors may be used to set an initial value and monitor whether each exercise results in an increase or decrease in perspiration. In some embodiments, image sensors used to, e.g., track facial expressions, eye movement, and/or facial reflexes may record initial values for comparison at different intervals and/or during portions of each VR exercise. In some embodiments, voice biomarkers may be used to track emotional states and/or determine intensity values for emotions.

In some embodiments, concurrently, as the user or patient starts or enters in the VR environment, biometric sensors start to measure and record biometric data of the patient for building biometric models for comparisons, diagnostics, and recommendations. For example, the initial biometric data may be used to build a baseline biometric model for comparison to data collected throughout the Cognitive Therapy session and especially for comparison at the end of the session. In addition, the collected data may be analyzed for various diagnoses as well as for recommendations for future activities, exercises, treatments, etc. In some cases, a patient may not be fully aware of how they are feeling. In some cases, a patient may perceive that they are not feeling good but may have difficulty identifying, e.g., more specifically how they feel until some biometric data, such as blood pressure or heart rate, is shown to them. FIG. 7 depicts a VR system with exemplary components of a VR therapy platform including several biometric sensors. Some embodiments may include sensors, such as eye movement tracking 1602, electroencephalogram (EEG) 1604, temperature sensor 1606, respiratory monitors 1608, microphone 1610, facial reflexive movement tracking 1612, facial expression monitoring 1614, electrocardiogram (EKG) 1616, blood pressure monitors 1618, perspiration sensor 1620, pulse oximeter monitor 1622, and cameras and light sensors 1624. The biometric sensors may measure and record a variety of biometric data including heart rate, respiration, temperature, perspiration, voice/speech (e.g., tone, intensity, pitch, etc.), eye movements, facial movements, mouth and jaw movements, hand and feet movements, neural and brain activities, etc., throughout the VR therapy session.

In some embodiments, biometric data may be used to correlate with the state of wellness of the patient at the start of the VR therapy session, throughout the exercises, and at the end of the session. For example, a therapist and/or patient may be able to help differentiate mental, physical, and/or emotional feelings or emotional states on a spectrum such as, e.g., feelings of depression, anxieties, frustrations, anger, rage, etc.

In some embodiments, biometric data may be used to supplement and/or adjust patient-reported data. For instance, in VR therapy, a patient may be down-playing or exaggerating an intensity level of an emotion or thought. Therapy generally works best when a patient is honest, but patients may not always be genuine and/or open to therapeutic assistance. Additional data may be used for comparison to patient-reported data to identify discrepancies and/or need for reconciliation. Some discrepancies may lead to adjustment of patient feedback data while some may be weighted or reconciled based on other patient data such as underlying conditions. Patient biometric data may be taken before, during, or at the end of a VR activity and used as a comparison. For instance, an initial intensity level for pain may be lowered based on a low(er) reading for a heart rate or perspiration level. In some cases, charts may be developed for therapists and doctors to observe discrepancies over time.

In some embodiments, biometric data may be used to supplement and/or adjust patient-reported data. For instance, in some embodiments, biometric values may be used in conjunction with patient input about emotional state and/or intensity values. In some embodiments, biometric data may be used to supplement and/or compare to patient survey data. For instance, using a VR assessment platform, a patient may take a survey that incorporates portions of the PHQ-9 (Patient Health Questionnaire-9), a multipurpose instrument for screening, diagnosing, monitoring, and measuring the severity of depression. Using the VR assessment platform and the PHQ-9, biometric data may be normalized and/or compared to responses and/or scores. In some embodiments, neural networks may be trained based on survey data and biometric data and used to determine if new biometric data may indicate a patient might relapse, stay steady, or improve. In some cases, biometrics and other feedback may validate whether a patient's status is improving, e.g., as indicated by surveys and assessments.

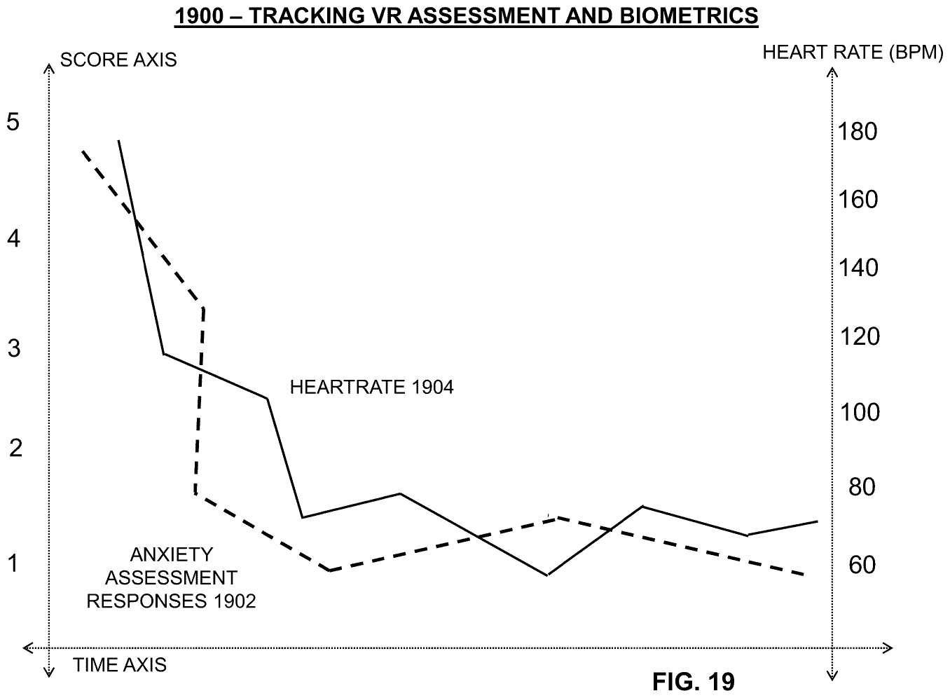

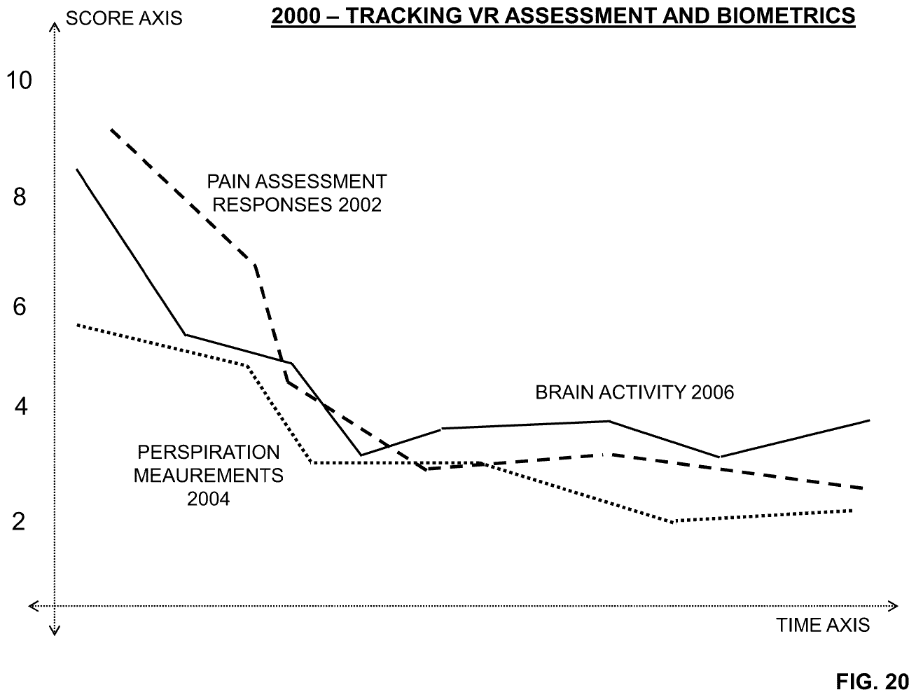

Generally, a VR engine may receive and record a biometric value at the beginning of a therapy session, at the end of therapy session, and/or during each of a plurality of VR activities. FIG. 19 is an illustrative chart depicting collected biometric data, heartrate data, in accordance with some embodiments of the present disclosure. FIG. 20 depicts an exemplary chart based on illustrative biometric measurements, e.g., brain activity and perspiration, recorded over time. With a successful therapy session, a chart like FIG. 19 or 20 will track biometric data depicting a patient's status improving, e.g., experiencing less intensity for one or more feelings emotions or calming down. If biometric data indicates more intense emotions, e.g., above a threshold such as 20% higher, another VR activity (or form of VR therapy) may be needed. In some embodiments, therapy exercises may affect a patient and her biometric data differently, but the end goal of VR therapy activities will be to achieve a measurement indicating that the exercises together will help improve the patient's status. For instance, a decline in a biometric data indicating more calmness—e.g., lower perspiration, lower heart rate, lower blood pressure, improved respiration, fewer involuntary movements, etc.—may not be achieved until after multiple VR activities.

In some embodiments, potential discrepancies in biometric data may be adjusted (or ignored) based on other factors such as the patient's conditions. For instance, motion sensors showing movement indicative of potential nervousness may be discounted if the patient has physical or mental issues causing tremors. Discrepancy data based on blood pressure spikes indicating high intensity emotion might be reduced if the patient is obese. Heart rate data indicating a very low BPM may not be a discrepancy if the patient is an athlete or otherwise in very good shape. Discrepancy data based on sound levels may be weighted differently if the patient has hearing issues. Respiratory illness may affect measurements by a pulse oximeter or respiratory sensors, which could imply a false discrepancy. Someone experiencing eye issues may have decreased eye movement and, accordingly, have a muted eye-movement measurement that may not corroborate a self-reported feeling such as nervousness, anxiety, worry, etc. Someone with chronic depression may experience lower blood pressure measurements.

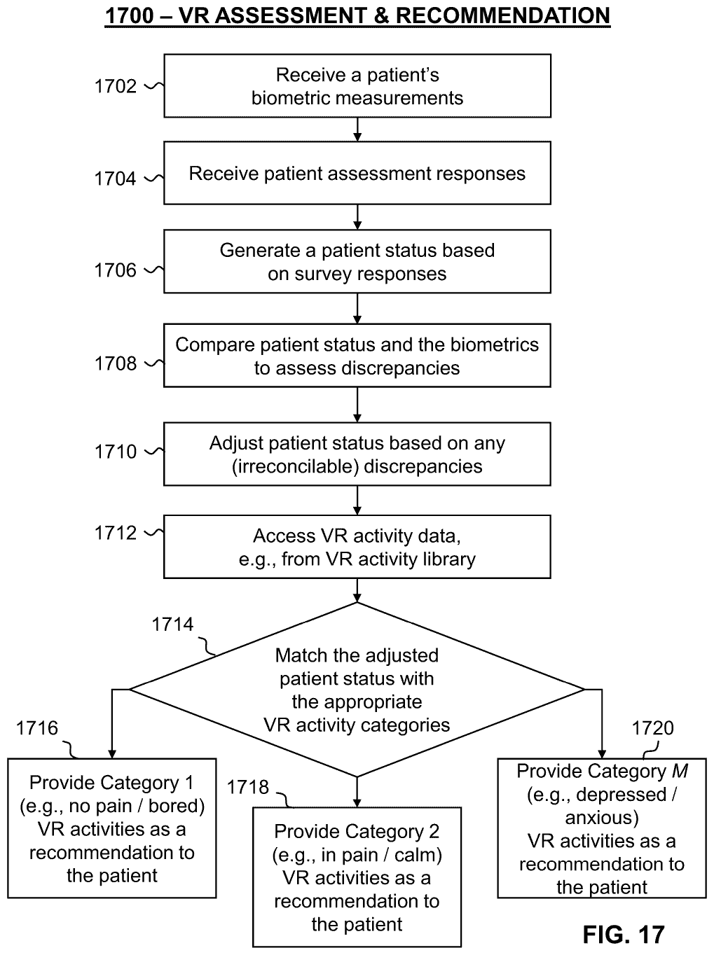

FIG. 17 depicts a flow chart of an exemplary process for VR assessment with biometric data, in accordance with embodiments of the present disclosure. VR assessment and recommendation may be carried out by one or more VR engines, e.g., as part of a VR platform running on VR hardware and/or network servers as depicted in FIGS. 21-24. At step 1702, a VR engine receives a patient's biometric measurements. At step 1704, the VR engine may receive patient assessment responses. At step 1706, the VR engine generates a patient status based on the survey responses. For instance, a patient status may be a category determined based on average scored in an assessment session, e.g., as depicted in FIGS. 12-14.

At step 1708, the VR engine may compare the patient status and the biometrics to, e.g., assess discrepancies between the patient status based on the survey responses and the patient's biometric measurements and determines whether happen any discrepancies are reconcilable, e.g., based on a patient's prior health history. At step 1710, the VR engine may adjust a patient status based on the (e.g., irreconcilable) discrepancies, e.g., based on the biometrics. At step 1712, the VR engine accesses data describing VR activities, e.g., in a VR activity library or database. In some embodiments, each of the VR activities will have a corresponding category or other data able to be matched with a patient status, e.g., to identify appropriate VR activities for such a categorized patient. For instance, VR assessment responses may be categorized and matched in accordance with process 1530 of FIG. 15B. In some embodiments, the VR engine may determine if VR activity metadata, e.g., titles, descriptions, categories, keywords, genres, styles, types, etc. matches characteristics associated with a patient status category.

At step 1714, the VR engine may match the adjusted patient status with the appropriate VR activity categories. For instance, if the patient status indicates, e.g., “no pain” and “bored” then, at step 1716, VR activities from category 1 may be provided. For example, in FIG. 11, the VR assessment platform suggests some appropriate VR activities such as trivia 1122, underwater adventures 1124, wildlife adventures 1126, and serene lake 1128. If the patient status indicates, e.g., “in pain” and “calm” then, at step 1718, VR activities from category 2 may be provided. There may be several categories, as the VR activity database may be finely categorized based on the patient status categories, in accordance with the patient responses from their assessments. For example, if the patient status indicates, e.g., “depressed” and “anxious” then, at step 1720, VR activities from category M may be provided.

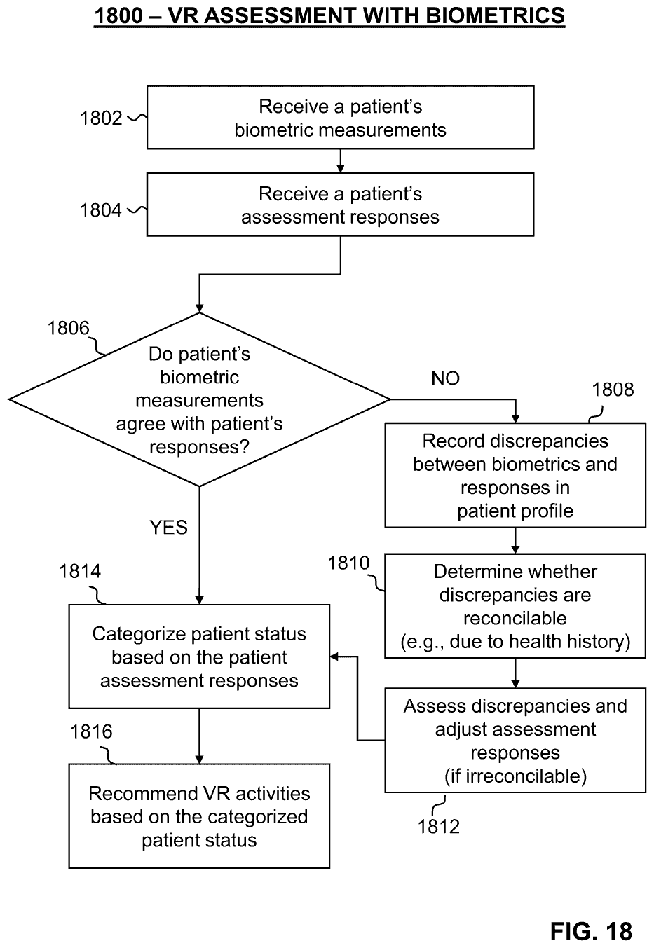

FIG. 18 depicts a flow chart of an exemplary process for VR assessment with biometric data, in accordance with embodiments of the present disclosure. VR assessment with biometrics may be carried out by one or more VR engines, e.g., as part of a VR platform running on VR hardware and/or network servers as depicted in FIGS. 21-24. Generally, A process for determining a discrepancy in patient-reported data may include steps for receiving a patient's biometric measurements, receiving a patient's input, comparing the biometric measurements to the input and determining whether there are any discrepancies in the patient's input. For instance, a patient may not be completely honest in some input, or unaware of subjectivity in his or her input, and a discrepancy in biometric feedback may highlight such an issue.

At step 1802, a VR engine may receive a patient's biometric measurements. At step 1804, the VR engine may receive a patient's assessment responses. At step 1806, the VR engine determines whether the patient's biometric measurements agree with patient's assessment responses.

If the VR engine determines the patient's biometric measurements do not agree with patient's assessment responses then, at step 1808, the discrepancies between the patient's biometric measurements and the patient's assessment responses are recorded in a patient profile. At step 1810, the VR engine determines whether the discrepancies are reconcilable, e.g., based on available patient health data. Some discrepancies may lead to adjustment of patient feedback data while some may be weighted or reconciled based on other patient data such as underlying conditions. For example, if a patient profile indicates a health history of hypertension, then biometric data about high blood pressure, e.g., indicating anxiety or stress or anxiety above the norm, may be considered reconcilable. For instance, if a patient profile indicates a health history of asthma, then biometric data about increased respiration, e.g., indicating pain or anxiety above the norm, may be considered reconcilable.

At step 1810, the VR engine assesses the discrepancies and may adjust the patient assessment responses, e.g., if irreconcilable. In some embodiments, if there is a discrepancy and there is no reason for complete reconciliation of the biometric data, the ledger data may be adjusted. For instance, if a (high) heart rate indicates a higher intensity value for, e.g., anxiety, the response data may be adjusted. If a (low) perspiration measurement indicates a lower intensity value for, e.g., anxiety, the response data may be adjusted accordingly, too. For example, a patient may report a high intensity value like “4” on a 0 to 4 scale for feeling anxiety but a measure of heart rate, blood pressure, brain activity, and/or perspiration may not corroborate such a high intensity value. For instance, an initial intensity level for pain level being 10 may be lowered based on a measured lower reading for a heart rate or perspiration level. In some cases, charts may be developed for therapists and doctors to observe discrepancies over time.

In some embodiments, response data may be adjusted without displaying the adjustment on the screen to avoid causing additional worry or confusion. For instance, someone self-reporting an intensity value of “5” for anxiety would probably not like to see an interface indicating that the VR engine decreased that intensity value to “4” based upon, e.g., a lower temperature, a lower heart rate, facial expressions, EKG, cameras, and/or other sensors. In some embodiments, the VR may provide the adjusted response data, e.g., to a therapist device. For example, it may be discouraging to show the patient that her response was adjusted. In some embodiments, the VR engine may provide to a therapist, e.g., via a therapist device, an indication that the patient-reported data was inaccurate. For instance, a patient may be exaggerating, underrepresenting, and/or lying about an intensity for an emotion, e.g., saying she feels an intensity level of “5” for anxiety, while her biometrics indicate a lesser intensity.

At step 1814, if the VR engine determines the patient's biometric measurements do agree with patient's assessment responses or after the assessment responses have been adjusted, the VR engine categorizes the patient status based on the assessment responses (e.g., unadjusted or adjusted, respectively). For instance, a patient status may be a category determined based on average scored in an assessment session, e.g., as depicted in FIGS. 12-14. In some embodiments, a category may be matched based on a process such as process 1530 of FIG. 15B. Then, at step 1816, the VR engine may recommend appropriate VR activities based on the categorized patient status.

FIG. 19 is an illustrative chart for collected biometric feedback during VR assessment, in accordance with some embodiments of the present disclosure. Chart 1900 of FIG. 19 depicts exemplary data based on an illustrative biometric measurement, e.g., beats per minute for a heartrate 1904, recorded over time as compared with anxiety assessment responses 1902. Generally, chart 1900 depicts assessment responses taken at regular intervals and heartrate measurements recorded at similar times. Data may be normalized to fit on the chart together and compare and monitor trends. Chart 1900 reflects improvement in both heartrate 1904 and anxiety assessment responses 1902, demonstrating, e.g., that the VR therapy may be helping to improve a patient's anxiety conditions. Such data may be collected by the VR therapy platform for evaluation. In some embodiments, a chart like chart 1900 may be presented to medical staff such as a doctor or therapist, e.g., via a therapist tablet. In some embodiments, such data may be recorded and used to train a predictive model to identify patient conditions based on biometrics.

FIG. 20 is an illustrative chart for collected biometric feedback during VR assessment, in accordance with some embodiments of the present disclosure. Chart 2000 of FIG. 20 depicts exemplary data based on an illustrative biometric measurement, e.g., brain activity 2006 and perspiration measurement 2004, recorded over time as compared with pain assessment responses 2002. Generally, chart 2000 depicts assessment responses taken at regular intervals and brain activity measurements and perspiration measurement recorded at similar times. Data may be normalized to fit on the chart together and compare and monitor trends. Chart 2000 reflects improvement in brain activity 2006, perspiration measurement 2004, and pain assessment responses 2002, demonstrating, e.g., that the VR therapy may be helping to improve a patient's pain conditions. Such data may be collected by the VR therapy platform for evaluation, e.g., to record data for analysis and/or provide further VR activity recommendations. In some embodiments, such data may be recorded and used to train a predictive model to identify patient conditions based on biometrics.

FIGS. 21A and 21B are diagrams of an illustrative system, in accordance with some embodiments of the disclosure. A VR system may include a clinician tablet 210, head-mounted display 201 (HMD or headset), small sensors 202, and large sensor 202B. Large sensor 202B may comprise transmitters, in some embodiments, and be referred to as wireless transmitter module 202B. Some embodiments may include sensor chargers, router, router battery, headset controller, power cords, USB cables, and other VR system equipment.

Clinician tablet 210 may be configured to use a touch screen, a power/lock button that turns the component on or off, and a charger/accessory port, e.g., USB-C. For instance, pressing the power button on clinician tablet 210 may power on the tablet or restart the tablet. Once clinician tablet 210 is powered on, a therapist or supervisor may access a user interface and be able to log in; add or select a patient; initialize and sync sensors; select, start, modify, or end a therapy session; view data; and/or log out.

Headset 201 may comprise a power button that turns the component on or off, as well as a charger/accessory port, e.g., USB-C. Headset 201 may also provide visual feedback of virtual reality applications in concert with the clinician tablet and the small and large sensors.

Charging headset 201 may be performed by plugging a headset power cord into the storage dock or an outlet. To turn on headset 201 or restart headset 201, the power button may be pressed. A power button may be on top of the headset. Some embodiments may include a headset controller used to access system settings. For instance, a headset controller may be used only in certain troubleshooting and administrative tasks and not necessarily during patient therapy. Buttons on the controller may be used to control power, connect to headset 201, access settings, or control volume.

The large sensor 202B (e.g., a wireless transmitter module) and small sensors 202 are equipped with mechanical and electrical components that measure position and orientation in physical space and then translate that information to construct a virtual environment. Sensors 202 are turned off and charged when placed in the charging station. Sensors 202 turn on and attempt to sync when removed from the charging station. The sensor charger may act as a dock to store and charge the sensors. In some embodiments, sensors may be placed in sensor bands on a patient. In some embodiments, sensors may be miniaturized and may be placed, mounted, fastened, or pasted directly onto a user.

As shown in illustrative FIG. 21A, various systems disclosed herein consist of a set of position and orientation sensors that are worn by a VR participant, e.g., a therapy patient. These sensors communicate with HMD 201, which immerses the patient in a VR experience. An HMD suitable for VR often comprises one or more displays to enable stereoscopic three-dimensional (3D) images. Such internal displays are typically high-resolution (e.g., 2880×1600 or better) and offer high refresh rate (e.g., 75 Hz). The displays are configured to present 3D images to the patient. VR headsets typically include speakers and microphones for deeper immersion.

HMD 201 is a piece central to immersing a patient in a virtual world in terms of presentation and movement. A headset may allow, for instance, a wide field of view (e.g., 110°) and tracking along six degrees of freedom. HMD 201 may include cameras, accelerometers, gyroscopes, and proximity sensors. VR headsets typically include a processor, usually in the form of a system on a chip (SoC), and memory. In some embodiments, headsets may also use, for example, additional cameras as safety features to help users avoid real-world obstacles. HMD 201 may comprise more than one connectivity option in order to communicate with the therapist's tablet. For instance, an HMD 201 may use an SoC that features WiFi and Bluetooth connectivity, in addition to an available USB connection (e.g., USB Type-C). The USB-C connection may also be used to charge the built-in rechargeable battery for the headset.

A supervisor, such as a health care provider or therapist, may use a tablet, e.g., tablet 210 depicted in FIG. 21A, to control the patient's experience. In some embodiments, tablet 210 runs an application and communicates with a router to cloud software configured to authenticate users and store information. Tablet 210 may communicate with HMD 201 in order to initiate HMD applications, collect relayed sensor data, and update records on the cloud servers. Tablet 210 may be stored in the portable container and plugged in to charge, e.g., via a USB plug.

In some embodiments, such as depicted in FIG. 21B, sensors 202 are placed on the body in particular places to measure body movement and relay the measurements for translation and animation of a VR avatar. Sensors 202 may be strapped to a body via bands 205. In some embodiments, each patient may have her own set of bands 205 to minimize hygiene issues.

A wireless transmitter module (WTM) 202B may be worn on a sensor band 205B that is laid over the patient's shoulders. WTM 202B sits between the patient's shoulder blades on their back. Wireless sensor modules 202 (e.g., sensors or WSMs) are worn just above each elbow, strapped to the back of each hand, and on a pelvis band that positions a sensor adjacent to the patient's sacrum on their back. In some embodiments, each WSM communicates its position and orientation in real-time with an HMD Accessory located on the HMD. Each sensor 202 may learn its relative position and orientation to the WTM, e.g., via calibration.

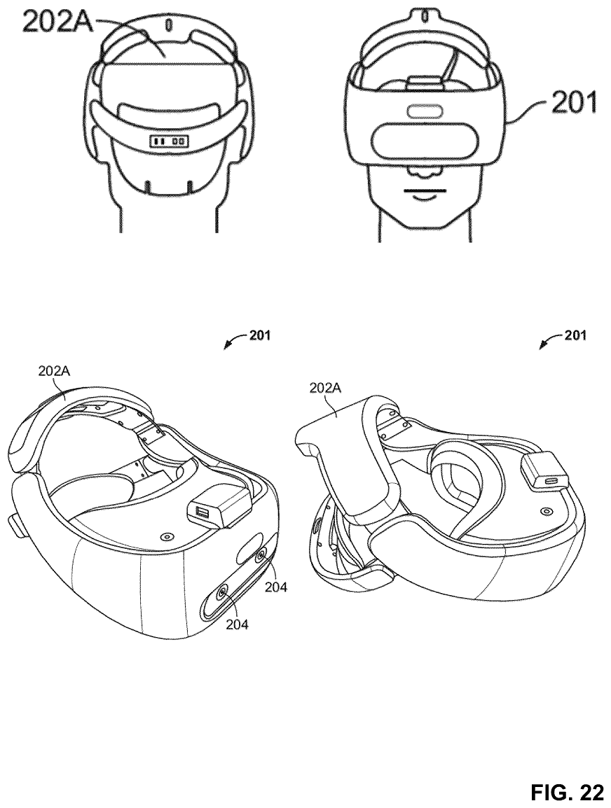

As depicted in FIG. 22, the HMD accessory may include a sensor 202A that may allow it to learn its position relative to WTM 202B, which then allows the HMD to know where in physical space all the WSMs and WTM are located. In some embodiments, each sensor 202 communicates independently with the HMD accessory which then transmits its data to HMD 201, e.g., via a USB-C connection. In some embodiments, each sensor 202 communicates its position and orientation in real-time with WTM 202B, which is in wireless communication with HMD 201. In some embodiments HMD 201 may be connected to input supplying other data such as biometric feedback data. For instance, in some cases, the VR system may include heart rate monitors, electrical signal monitors, e.g., electrocardiogram (EKG), eye movement tracking, brain monitoring with Electroencephalogram (EEG), pulse oximeter monitors, temperature sensors, blood pressure monitors, respiratory monitors, light sensors, cameras, sensors, and other biometric devices. Biometric feedback, along with other performance data, can indicate more subtle changes to the patient's body or physiology as well as mental state, e.g., when a patient is stressed, comfortable, distracted, tired, over-worked, under-worked, over-stimulated, confused, overwhelmed, excited, engaged, disengaged, and more. In some embodiments, such devices measuring biometric feedback may be connected to the HMD and/or the supervisor tablet via USB, Bluetooth, Wi-Fi, radio frequency, and other mechanisms of networking and communication.

A VR environment rendering engine on HMD 201 (sometimes referred to herein as a “VR application”), such as the Unreal Engine™, uses the position and orientation data to create an avatar that mimics the patient's movement.

A patient or player may “become” their avatar when they log in to a virtual reality activity. When the player moves their body, they see their avatar move accordingly. Sensors in the headset may allow the patient to move the avatar's head, e.g., even before body sensors are placed on the patient. A system that achieves consistent high-quality tracking facilitates the patient's movements to be accurately mapped onto an avatar.

Sensors 202 may be placed on the body, e.g., of a patient by a therapist, in particular locations to sense and/or translate body movements. The system can use measurements of position and orientation of sensors placed in key places to determine movement of body parts in the real world and translate such movement to the virtual world. In some embodiments, a VR system may collect performance data for therapeutic analysis of a patient's movements and range of motion.

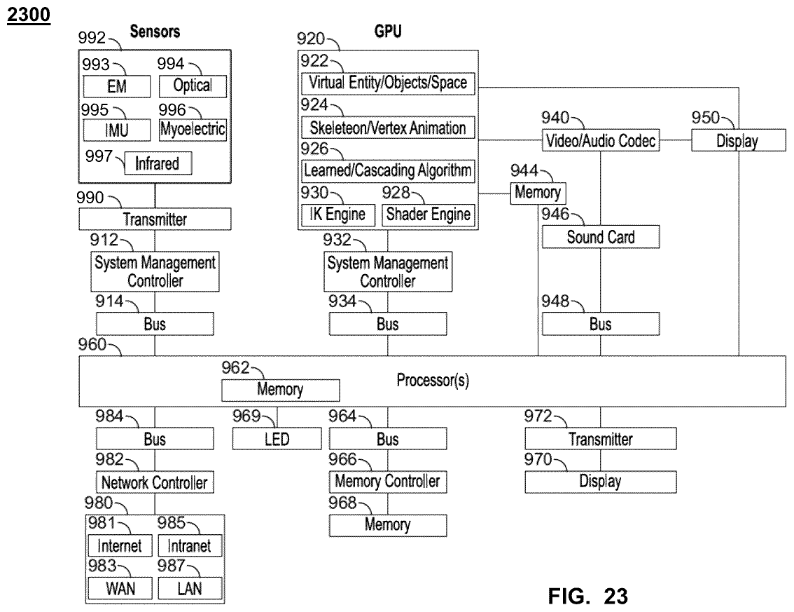

In some embodiments, systems and methods of the present disclosure may use electromagnetic tracking, optical tracking, infrared tracking, accelerometers, magnetometers, gyroscopes, myoelectric tracking, other tracking techniques, or a combination of one or more of such tracking methods. The tracking systems may be parts of a computing system as disclosed herein. The tracking tools may exist on one or more circuit boards within the VR system (see FIG. 23) where they may monitor one or more users to perform one or more functions such as capturing, analyzing, and/or tracking a subject's movement. In some cases, a VR system may utilize more than one tracking method to improve reliability, accuracy, and precision.

FIG. 23 depicts an illustrative arrangement for various elements of a system, e.g., an HMD and sensors of FIGS. 21A-B and FIG. 22. The arrangement includes one or more printed circuit boards (PCBs). In general terms, the elements of this arrangement track, model, and display a visual representation of the participant (e.g., a patient avatar) in the VR world by running software including the aforementioned VR application of HMD 201.

The arrangement shown in FIG. 23 includes one or more sensors 992, processors 960, graphic processing units (GPUs) 920, video encoder/video codec 940, sound cards 946, transmitter modules 990, network interfaces 980, and light emitting diodes (LEDs) 969. These components may be housed on a local computing system or may be remote components in wired or wireless connection with a local computing system (e.g., a remote server, a cloud, a mobile device, a connected device, etc.). Connections between components may be facilitated by one or more buses, such as bus 914, bus 934, bus 948, bus 984, and bus 964 (e.g., peripheral component interconnects (PCI) bus, PCI-Express bus, or universal serial bus (USB)). With such buses, the computing environment may be capable of integrating numerous components, numerous PCBs, and/or numerous remote computing systems.

One or more system management controllers, such as system management controller 912 or system management controller 932, may provide data transmission management functions between the buses and the components they integrate. For instance, system management controller 912 provides data transmission management functions between bus 914 and sensors 992. System management controller 932 provides data transmission management functions between bus 934 and GPU 920. Such management controllers may facilitate the arrangements orchestration of these components that may each utilize separate instructions within defined time frames to execute applications. Network interface 980 may include an ethernet connection or a component that forms a wireless connection, e.g., 802.11b, g, a, or n connection (WiFi), to a local area network (LAN) 987, wide area network (WAN) 983, intranet 985, or internet 981. Network controller 982 provides data transmission management functions between bus 984 and network interface 980.

A device may receive content and data via input/output (hereinafter “I/O”) path. I/O path may provide content (e.g., content available over a local area network (LAN) or wide area network (WAN), and/or other content) and data to control circuitry 1204, which includes processing circuitry 1206 and storage 1208. Control circuitry may be used to send and receive commands, requests, and other suitable data using I/O path. I/O path may connect control circuitry (and processing circuitry) to one or more communications paths. I/O functions may be provided by one or more of these communications paths.

Control circuitry may be based on any suitable processing circuitry such as processing circuitry. As referred to herein, processing circuitry should be understood to mean circuitry based on one or more microprocessors, microcontrollers, digital signal processors, programmable logic devices, field-programmable gate arrays (FPGAs), application-specific integrated circuits (ASICs), etc., and may include a multi-core processor (e.g., dual-core, quad-core, hexa-core, or any suitable number of cores). In some embodiments, processing circuitry may be distributed across multiple separate processors or processing units, for example, multiple of the same type of processing units (e.g., two Intel Core i7 processors) or multiple different processors (e.g., an Intel Core i5 processor and an Intel Core i7 processor). In some embodiments, control circuitry executes instructions for receiving streamed content and executing its display, such as executing application programs that provide interfaces for content providers to stream and display content on a display.

Control circuitry may thus include communications circuitry suitable for communicating with a content provider server or other networks or servers. Communications circuitry may include a cable modem, an integrated services digital network (ISDN) modem, a digital subscriber line (DSL) modem, a telephone modem, Ethernet card, or a wireless modem for communications with other equipment, or any other suitable communications circuitry. Such communications may involve the Internet or any other suitable communications networks or paths. In addition, communications circuitry may include circuitry that enables peer-to-peer communication of user equipment devices, or communication of user equipment devices in locations remote from each other.

Processor(s) 960 and GPU 920 may execute a number of instructions, such as machine-readable instructions. The instructions may include instructions for receiving, storing, processing, and transmitting tracking data from various sources, such as electromagnetic (EM) sensors 993, optical sensors 994, infrared (IR) sensors 997, inertial measurement units (IMUs) sensors 995, and/or myoelectric sensors 996. The tracking data may be communicated to processor(s) 960 by either a wired or wireless communication link, e.g., transmitter 990. Upon receiving tracking data, processor(s) 960 may execute an instruction to permanently or temporarily store the tracking data in memory 962 such as, e.g., random access memory (RAM), read only memory (ROM), cache, flash memory, hard disk, or other suitable storage component. Memory may be a separate component, such as memory 968, in communication with processor(s) 960 or may be integrated into processor(s) 960, such as memory 962, as depicted.

Memory may be an electronic storage device provided as storage that is part of control circuitry. As referred to herein, the phrase “electronic storage device” or “storage device” should be understood to mean any device for storing electronic data, computer software, or firmware, such as random-access memory, read-only memory, hard drives, optical drives, digital video disc (DVD) recorders, compact disc (CD) recorders, BLU-RAY disc (BD) recorders, BLU-RAY 3D disc recorders, digital video recorders (DVR, sometimes called a personal video recorder, or PVR), solid state devices, quantum storage devices, gaming consoles, gaming media, or any other suitable fixed or removable storage devices, and/or any combination of the same. Storage may be used to store various types of content described herein as well as media guidance data described above. Nonvolatile memory may also be used (e.g., to launch a boot-up routine and other instructions). Cloud-based storage may be used to supplement storage or instead of storage.

Storage may also store instructions or code for an operating system and any number of application programs to be executed by the operating system. In operation, processing circuitry retrieves and executes the instructions stored in storage, to run both the operating system and any application programs started by the user. The application programs can include one or more voice interface applications for implementing voice communication with a user, and/or content display applications which implement an interface allowing users to select and display content on display or another display.

Processor(s) 960 may also execute instructions for constructing an instance of virtual space. The instance may be hosted on an external server and may persist and undergo changes even when a participant is not logged in to said instance. In some embodiments, the instance may be participant-specific, and the data required to construct it may be stored locally. In such an embodiment, new instance data may be distributed as updates that users download from an external source into local memory. In some exemplary embodiments, the instance of virtual space may include a virtual volume of space, a virtual topography (e.g., ground, mountains, lakes), virtual objects, and virtual characters (e.g., non-player characters “NPCs”). The instance may be constructed and/or rendered in 2D or 3D. The rendering may offer the viewer a first-person or third-person perspective. A first-person perspective may include displaying the virtual world from the eyes of the avatar and allowing the patient to view body movements from the avatar's perspective. A third-person perspective may include displaying the virtual world from, for example, behind the avatar to allow someone to view body movements from a different perspective. The instance may include properties of physics, such as gravity, magnetism, mass, force, velocity, and acceleration, which cause the virtual objects in the virtual space to behave in a manner at least visually similar to the behaviors of real objects in real space.

Processor(s) 960 may execute a program (e.g., the Unreal Engine or VR applications discussed above) for analyzing and modeling tracking data. For instance, processor(s) 960 may execute a program that analyzes the tracking data it receives according to algorithms described above, along with other related pertinent mathematical formulas. Such a program may incorporate a graphics processing unit (GPU) 920 that is capable of translating tracking data into 3D models. GPU 920 may utilize shader engine 928, vertex animation 924, and linear blend skinning algorithms. In some instances, processor(s) 960 or a CPU may at least partially assist the GPU in making such calculations. This allows GPU 920 to dedicate more resources to the task of converting 3D scene data to the projected render buffer. GPU 920 may refine the 3D model by using one or more algorithms, such as an algorithm learned on biomechanical movements, a cascading algorithm that converges on a solution by parsing and incrementally considering several sources of tracking data, an inverse kinematics (IK) engine 930, a proportionality algorithm, and other algorithms related to data processing and animation techniques. After GPU 920 constructs a suitable 3D model, processor(s) 960 executes a program to transmit data for the 3D model to another component of the computing environment (or to a peripheral component in communication with the computing environment) that is capable of displaying the model, such as display 950.

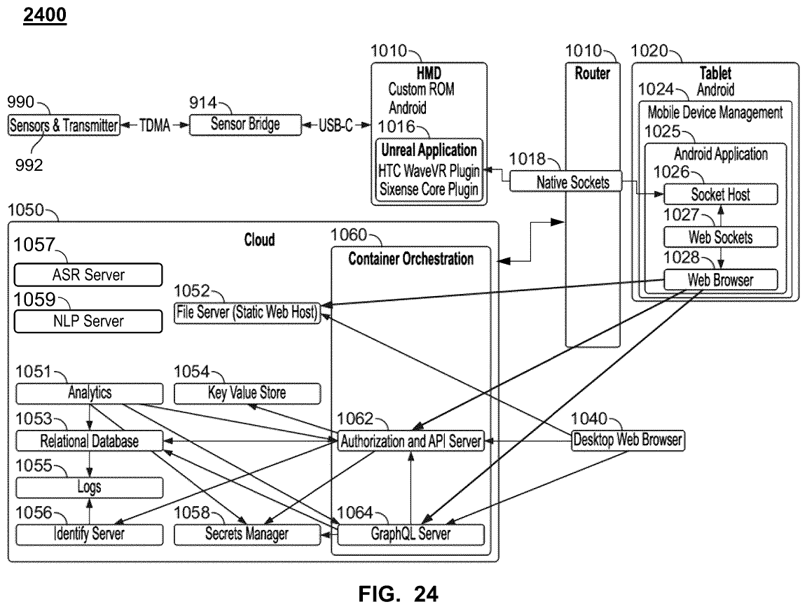

In some embodiments, GPU 920 transfers the 3D model to a video encoder or a video codec 940 via a bus, which then transfers information representative of the 3D model to a suitable display 950. The 3D model may be representative of a virtual entity that can be displayed in an instance of virtual space, e.g., an avatar. The virtual entity is capable of interacting with the virtual topography, virtual objects, and virtual characters within virtual space. The virtual entity is controlled by a user's movements, as interpreted by sensors 992 communicating with the system. Display 950 may display a Patient View. The patient's real-world movements are reflected by the avatar in the virtual world. The virtual world may be viewed in the headset in 3D and monitored on the tablet in two dimensions. In some embodiments, the VR world is an activity that provides feedback and rewards based on the patient's ability to complete activities. Data from the in-world avatar is transmitted from the HMD to the tablet to the cloud, where it is stored for later analysis. An illustrative architectural diagram of such elements in accordance with some embodiments is depicted in FIG. 24.

A VR system may also comprise display 970, which is connected to the computing environment via transmitter 972. Display 970 may be a component of a clinician tablet. For instance, a supervisor or operator, such as a therapist, may securely log in to a clinician tablet, coupled to the system, to observe and direct the patient to participate in various activities and adjust the parameters of the activities to best suit the patient's ability level. Display 970 may depict a view of the avatar and/or replicate the view of the HMD.

In some embodiments, HMD 201 may be the same as or similar to HMD 1010 in FIG. 24. In some embodiments, HMD 1010 runs a version of Android that is provided by HTC (e.g., a headset manufacturer) and the VR application is an Unreal application, e.g., Unreal Application 1016, encoded in an Android package (.apk). The .apk comprises a set of custom plugins: WVR, WaveVR, SixenseCore, SixenseLib, and MVICore. The WVR and WaveVR plugins allow the Unreal application to communicate with the VR headset's functionality. The SixenseCore, SixenseLib, and MVICore plugins allow Unreal Application 1016 to communicate with the HMD accessory and sensors that communicate with the HMD via USB-C. The Unreal Application comprises code that records the position and orientation (PnO) data of the hardware sensors and translates that data into a patient avatar, which mimics the patient's motion within the VR world. An avatar can be used, for example, to infer and measure the patient's real-world range of motion. The Unreal application of the HMD includes an avatar solver as described, for example, below.

The clinician operator device, clinician tablet 1020, runs a native application (e.g., Android application 1025) that allows an operator such as a therapist to control a patient's experience. Cloud server 1050 includes a combination of software that manages authentication, data storage and retrieval, and hosts the user interface, which runs on the tablet. This can be accessed by tablet 1020. Tablet 1020 has several modules.

As depicted in FIG. 24, the first part of tablet software is a mobile device management (MDM) 1024 layer, configured to control what software runs on the tablet, enable/disable the software remotely, and remotely upgrade the tablet applications.

The second part is an application, e.g., Android Application 1025, configured to allow an operator to control the software of HMD 1010. In some embodiments, the application may be a native application. A native application, in turn, may comprise two parts, e.g., (1) socket host 1026 configured to receive native socket communications from the HMD and translate that content into web sockets, e.g., web sockets 1027, that a web browser can easily interpret; and (2) a web browser 1028, which is what the operator sees on the tablet screen. The web browser may receive data from the HMD via the socket host 1026, which translates the HMD's native socket communication 1018 into web sockets 1027, and it may receive UI/UX information from a file server 1052 in cloud 1050. Tablet 1020 comprises web browser 1028, which may incorporate a real-time 3D engine, such as Babylon.js, using a JavaScript library for displaying 3D graphics in web browser 1028 via HTML5. For instance, a real-time 3D engine, such as Babylon.js, may render 3D graphics, e.g., in web browser 1028 on clinician tablet 1020, based on received skeletal data from an avatar solver in the Unreal Engine 1016 stored and executed on HMD 1010. In some embodiments, rather than Android Application 1026, there may be a web application or other software to communicate with file server 1052 in cloud 1050. In some instances, an application of Tablet 1020 may use, e.g., Web Real-Time Communication (WebRTC) to facilitate peer-to-peer communication without plugins, native apps, and/or web sockets.

The cloud software, e.g., cloud 1050, has several different, interconnected parts configured to communicate with the tablet software: authorization and API server 1062, GraphQL server 1064, and file server (static web host) 1052.

In some embodiments, authorization and API server 1062 may be used as a gatekeeper. For example, when an operator attempts to log in to the system, the tablet communicates with the authorization server. This server ensures that interactions (e.g., queries, updates, etc.) are authorized based on session variables such as operator's role, the health care organization, and the current patient. This server, or group of servers, communicates with several parts of the system: (a) a key value store 1054, which is a clustered session cache that stores and allows quick retrieval of session variables; (b) a GraphQL server 1064, as discussed below, which is used to access the back-end database in order to populate the key value store, and also for some calls to the application programming interface (API); (c) an identity server 1056 for handling the user login process; and (d) a secrets manager 1058 for injecting service passwords (relational database, identity database, identity server, key value store) into the environment in lieu of hard coding.

When the tablet requests data, it will communicate with the GraphQL server 1064, which will, in turn, communicate with several parts: (1) the authorization and API server 1062; (2) the secrets manager 1058, and (3) a relational database 1053 storing data for the system. Data stored by the relational database 1053 may include, for instance, profile data, session data, application data, activity performance data, and motion data.

In some embodiments, profile data may include information used to identify the patient, such as a name or an alias. Session data may comprise information about the patient's previous sessions, as well as, for example, a “free text” field into which the therapist can input unrestricted text, and a log 1055 of the patient's previous activity. Logs 1055 are typically used for session data and may include, for example, total activity time, e.g., how long the patient was actively engaged with individual activities; activity summary, e.g., a list of which activities the patient performed, and how long they engaged with each on; and settings and results for each activity. Activity performance data may incorporate information about the patient's progression through the activity content of the VR world. Motion data may include specific range-of-motion (ROM) data that may be saved about the patient's movement over the course of each activity and session, so that therapists can compare session data to previous sessions' data.

In some embodiments, file server 1052 may serve the tablet software's website as a static web host.

Cloud server 1050 may also include one or more systems for implementing processes of voice processing in accordance with embodiments of the disclosure. For instance, such a system may perform voice identification/differentiation, determination of interrupting and supplemental comments, and processing of voice queries. A computing device may be in communication with an automated speech recognition (ASR) server 1057 through, for example, a communications network. ASR server 1057 may also be in electronic communication with natural language processing (NLP) server 1059 also through, for example, a communications network. ASR server 1057 and/or NLP server 1059 may be in communication with one or more computing devices running a user interface, such as a voice assistant, voice interface allowing for voice-based communication with a user, or an electronic content display system for a user. Examples of such computing devices are a smart home assistant similar to a Google Home® device or an Amazon® Alexa® or Echo® device, a smartphone or laptop computer with a voice interface application for receiving and broadcasting information in voice format, a set-top box or television running a media guide program or other content display program for a user, or a server executing a content display application for generating content for display to a user. ASR server 1057 may be any server running an ASR application. NLP server 1059 may be any server programmed to process one or more voice inputs in accordance with embodiments of the disclosure, and to process voice queries with the ASR server 1057. In some embodiments, one or more of ASR server 1057 and NLP server 1059 may be components of cloud server 1050 depicted in FIG. 24. In some embodiments, a form of one or more of ASR server 1057 and NLP server 1059 may be components of HMD 201.

While the foregoing discussion describes exemplary embodiments of the present invention, one skilled in the art will recognize from such discussion, the accompanying drawings, and the claims, that various modifications can be made without departing from the spirit and scope of the invention. Therefore, the illustrations and examples herein should be construed in an illustrative, and not a restrictive sense. The scope and spirit of the invention should be measured solely by reference to the claims that follow.

1. A method of recommending a virtual reality (VR) activity based on assessment responses by a patient, the method comprising:
receiving a plurality of assessment responses concerning mental or physical conditions of the patient;
determining a patient status category based on the plurality of assessment responses;
accessing data describing a plurality of VR activities;
selecting a subset of VR activities appropriate for the mental or physical conditions of the patient from the plurality of VR activities based on the patient status category and data describing the plurality of VR activities; and
providing the subset of VR activities as a recommendation.

2. The method of claim 1, wherein the mental or physical conditions comprise at least one selected from the following: depression, anxiety, and pain.

3. The method of claim 1, wherein each of the plurality of assessment responses comprises an assessment score; and
determining the patient status category based on the plurality of assessment responses comprises:
generating, based on at least a portion of the plurality of assessment responses comprising the assessment scores, a condition scale score for at least one of the mental or physical conditions of the patient; and
determining the patient status category based on the condition scale score.

4. The method of claim 1, wherein the selecting the subset of the plurality of VR activities based on the patient status category and data describing the plurality of VR activities comprises:
comparing the patient status category to data describing each of the plurality of VR activities;
generating a match score for each of the plurality of VR activities, based on the comparing of the patient status category to the data describing each of the plurality of VR activities; and
selecting a subset of the plurality of VR activities based on the respective match score of each of the subset of the plurality of VR activities.

5. The method of claim 1 further comprising:
receiving a second plurality of assessment responses concerning mental or physical conditions of a patient;
updating the patient status category based on the plurality of assessment responses;
selecting a second subset of VR activities appropriate for the patient from the plurality of VR activities based on the patient status category and the metadata of each of the plurality of VR activities; and
providing the second subset of VR activities as a recommendation.

6. The method of claim 1, wherein the receiving the plurality of assessment responses associated with a patient further comprises receiving a biometric measurement of the patient and adjusting one or more of the plurality of assessment responses based on the received biometric measurements.

7. The method of claim 1, wherein the receiving the plurality of assessment responses associated with a patient further comprises:
receiving a biometric measurement for the patient associated with at least one of the plurality of assessment responses;
normalizing the biometric measurement;
determining a discrepancy between the normalized biometric measurement and the at least one of the plurality of assessment responses; and
recording the discrepancy between the normalized biometric measurement and the at least one of the plurality of assessment responses.

8. The method of claim 7 further comprising:
in response to determining the discrepancy between the normalized biometric measurement and the at least one of the plurality of assessment responses is greater than a predetermined threshold, adjusting the at least one of the plurality of assessment responses based on the normalized biometric measurement.

9. The method of claim 7 further comprising:
in response to determining the discrepancy between the normalized biometric measurement and the at least one of the plurality of assessment responses is greater than a predetermined threshold:
accessing a patient profile for the patient;
determining whether the patient profile includes one or more reconciliatory conditions related to the biometric measurement;
in response to determining the determining the patient profile does not include one or more reconciliatory conditions, adjusting the at least one of the plurality of assessment responses based on the normalized biometric measurement; and
in response to determining the determining the patient profile includes one or more reconciliatory conditions, providing the at least one of the plurality of assessment responses without adjustment based on the biometric measurement.

10. The method of claim 7, wherein the biometric measurement is transmitted from at least one of the following: an eye movement tracker, an electroencephalogram (EEG), a temperature sensor, a respiratory monitor, a microphone, a facial reflexive movement tracker, a facial expression monitor, an electrocardiogram (EKG), a blood pressure monitor, a perspiration sensor, a pulse oximeter monitor, a camera, and a light sensor.

11. A method of recommending a virtual reality (VR) therapy activity, the method comprising:
providing, by the VR platform, a plurality of prompts requesting a plurality of responses for a condition of a patient;
receiving the plurality of responses as input from the patient;
calculating an assessment response value based on the plurality of responses;
receiving a biometric measurement for the patient related to the condition of the patient;
accessing data describing a plurality of VR activities;
determining a discrepancy between the biometric measurement and the normalized intensity score;
in response to determining the discrepancy between the biometric measurement and the assessment response value is greater than a predetermined threshold, adjusting the assessment response value based on the biometric measurement;
generating a patient status category based on the adjusted assessment response value;
selecting a subset of the plurality of VR activities based on the patient status category and the data describing the plurality of VR activities; and
providing the subset as a recommendation.

12. The method of claim 11, wherein calculating the assessment response value based on the plurality of responses comprises calculating, for at least a portion of the plurality of responses, at least one selected from the group consisting essentially of a mean, a median, mode, maximum, minimum, and a weighted average.

13. The method of claim 11 further comprising:
in response to determining the discrepancy between the biometric measurement and the assessment response value is not greater than a predetermined threshold, generating the patient status category based on the response value; and
providing the subset as a recommendation.

14. The method of claim 11, wherein the receiving the biometric measurement for the patient further comprises normalizing the biometric measurement.

15. The method of claim 11, wherein adjusting the response value based on the biometric measurement further comprises:
accessing a patient profile for the patient;
determining whether the patient profile includes one or more reconciliatory conditions related to the biometric measurement;
in response to determining that the patient profile does not include one or more reconciliatory conditions, adjusting the at least one of the plurality of assessment responses based on the biometric measurement; and
in response to determining that the patient profile includes one or more reconciliatory conditions, providing the at least one of the plurality of assessment responses without adjustment based on the biometric measurement.

16. The method of claim 15, wherein the patient profile for the patient comprises one or more conditions that may affect one or more biometric measurements.

17. The method of claim 11, wherein the biometric measurement is selected from one of the following: heart rate, respiration, temperature, perspiration, voice tone, voice intensity, voice pitch, eye movement, facial movement, mouth movement, jaw movement, hand movement, feet movement, neural activities, and brain activities.

18. The method of claim 11, wherein the biometric measurement is transmitted from at least one selected from the following: an eye movement tracker, an electroencephalogram (EEG), a temperature sensor, a respiratory monitor, a microphone, a facial reflexive movement tracker, a facial expression monitor, an electrocardiogram (EKG), a blood pressure monitor, a perspiration sensor, a pulse oximeter monitor, a camera, and a light sensor.

19. The method of claim 11, wherein the discrepancy is recorded in a data structure to be provided in a user interface.

20. The method of claim 11 further comprising providing, by the VR platform, a second plurality of prompts requesting a second plurality of responses for the condition of the patient at a predetermined interval.

21-40. (canceled)
